(19) United States
(12) Patent Application Publication
(10) Pub. No.: US 2024/0382520 A1
(43) Pub. Date: Nov. 21, 2024

(54) COMPOSITION FOR PREVENTING AND TREATING CANCER THROUGH MITOCHONDRIAL METABOLISM REGULATION CONTAINING NANOGRAPHENE STRUCTURE

(71) Applicants: SEOUL NATIONAL UNIVERSITY R & DB FOUNDATION, Seoul (KR); INBCT CO., LTD., Seoul (KR)

(72) Inventors: Kyung-Sun KANG, Seoul (KR); Da-Hyun KIM, Hanam-si (KR); Jaechul RYU, Uiwang-si (KR)

(73) Assignees: SEOUL NATIONAL UNIVERSITY R & DB FOUNDATION, Seoul (KR); INBCT CO., LTD., Seoul (KR)

(21) Appl. No.: 18/684,650

(22) PCT Filed: Aug. 22, 2022
(86) PCT No.: PCT/KR2022/012549

§ 371 Date: Mar. 1, 2024

(30) Foreign Application Priority Data
Aug. 20, 2021 (KR) 10-2021-0110275
Aug. 22, 2022 (KR) 10-2022-0105084

(51) Int. Cl. A61K 33/44; A61K 9/14; A61K 47/10; A61K 47/18; A61P 35/00

(52) U.S. Cl. CPC A61K 33/44; A61K 9/14; A61K 47/10; A61K 47/18; A61P 35/00

(57) ABSTRACT

The present invention pertains to a composition for preventing and treating cancer through mitochondrial metabolism regulation, wherein the composition contains a nanographene structure. It has been confirmed that a specific inhibitory activity on the NIX-dependent pathway among the mitophagy mechanisms in cancer cells inhibits mitochondrial degradation, and thus damaged mitochondria accumulate in the cancer cells, and apoptosis of the cancer cells is induced as a result. Therefore, the composition according to the present invention exhibits excellent anticancer effects that can be applied to various diseases related to the accumulation of abnormal cells, without causing resistance or toxicity, and can thus be effectively used as a cancer prevention or treatment agent or an anticancer treatment adjuvant.

This application is a U.S. National Stage of International Patent Application No. PCT/KR2022/012549, filed Aug. 22, 2022, which claims the benefit of priority to Korean Patent Application No. 10-2021-0110275, filed on Aug. 20, 2021, and Korean Patent Application No. 10-2022-0105084, filed on Aug. 22, 2022, the contents of which are incorporated herein by reference in their entireties.

### TECHNICAL FIELD

The present invention relates to a composition for preventing and treating cancer through the regulation of mitochondrial metabolism, including a nanographene structure.

The instant application contains Sequence Listing which has been submitted electronically in XML format and is hereby incorporated in its entirety. Said XML copy, created on 2024 Feb. 19, is named Sequence Listing.

### BACKGROUND ART

Mitochondria, which are at the center of metabolic processes in the cell, not only produce energy by oxidative phosphorylation, but also perform essential functions for maintaining the life of cells and organisms, such as apoptosis, ion homeostasis, sugar and fat metabolism (intermediary metabolism), pyrimidine synthesis, urea and heme synthesis and the like, and particularly, since they are associated with energy production, scheduled cell death and the generation of reactive oxygen species (ROS), they are involved at various times in various diseases, such as cancer, diabetes, neurodegenerative diseases, aging and the like.

Mitochondrial activity is maintained by the balance of mitochondrial generation, fusion, fission mechanisms and mitophagy activity. Mitophagy is a type of autophagy, and it is a decomposition mechanism that selectively removes unnecessary mitochondria due to aging or damage. When mitophagy occurs, a membrane is formed, and inside the membrane, mitochondria are fused with lysosomes and completely degraded.

The mitophagy mechanism, which is a type of autophagy, can be induced in stressed cells as a cell survival mechanism, and although it suppresses tumors before cancer progresses and during cancer development, it contributes to the survival of tumor cells after cancer is generated, and thus, when autophagy is suppressed in tumor cells where cancer is generated, it may have the effect of reducing the activity of tumor cells.

Meanwhile, graphene is a nanomaterial made of carbon and has been mainly used as a drug delivery system (DDS) for active ingredients, but recently, research is being conducted to confirm the effects of graphene itself. For example, it has been reported that graphene can damage mitochondria and kill cancer cells through the generation of ROS.

However, there has been no report on cancer prevention or treatment technology which confirms the effects of nanographene in relation to the inhibition of mitophagy and utilizes the same.

### DISCLOSURE

### Technical Problem

In order to solve the above problems, the composition including nanographene oxide or a modified structure thereof as an active ingredient was completed.

An object of the present invention is to provide a pharmaceutical composition for preventing or treating cancer, a pharmaceutical composition for supplementing anticancer therapy, a food composition for preventing or ameliorating cancer, a composition for inhibiting mitophagy and a composition for apoptosis, including a nanographene structure as an active ingredient.

However, the technical problems to be achieved by the present invention are not limited to the above-mentioned problems, and other problems that are not mentioned will be clearly understood by those skilled in the art from the description below.

### Technical Solution

The present invention provides a pharmaceutical composition for preventing or treating cancer, a pharmaceutical composition for supplementing anticancer therapy, a food composition for preventing or ameliorating cancer, a composition for inhibiting mitophagy and a composition for apoptosis, including a nanographene structure as an active ingredient.

In an exemplary embodiment of the present invention, the nanographene structure may be any one selected from the group consisting of a nanographene oxide, a nanographene oxide variant and graphene quantum dots, but the present invention is not limited thereto.

In an exemplary embodiment of the present invention, the nanographene oxide may exhibit any one or more of the following characteristics, but the present invention is not limited thereto:
1) it has a circular plate shape;
2) as a result of Raman spectroscopy analysis, it has a carbon-derived material-specific Raman shift; and
3) as a result of Raman spectroscopy analysis, graphene active groups —OH, —C—H and C═O are confirmed.

In an exemplary embodiment of the present invention, the nanographene oxide variant may be any one of a PEGylated nanographene oxide or an aminated nanographene oxide in which PEG (polyethylene glycol) or NH₂ (amino group) is bonded to a nanographene oxide, but the present invention is not limited thereto

In an exemplary embodiment of the present invention, the PEGylated nanographene oxide may exhibit any one or more of the following characteristics, but the present invention is not limited thereto:
1) the zeta potential of graphene ranges from −25.0 to −21.0 mV; and
2) it has a particle size of approximately 10 nm.

In an exemplary embodiment of the present invention, the aminated nanographene oxide may exhibit the following characteristic, but the present invention is not limited thereto:
1) it has a diameter of 24.4 to 38.8 nm.

In an exemplary embodiment of the present invention, the nanographene structure may inhibit mitochondrial mitophagy, but the present invention is not limited thereto.

In an exemplary embodiment of the present invention, the nanographene structure may specifically inhibit NIX, but the present invention is not limited thereto.

In an exemplary embodiment of the present invention, the nanographene structure may not inhibit BNIP3 and PINK1, but the present invention is not limited thereto.

In an exemplary embodiment of the present invention, the nanographene structure may be included at a concentration of 0.01 to 200 μg/mL, but the present invention is not limited thereto.

In an exemplary embodiment of the present invention, the cancer may be one or more selected from the group consisting of thyroid cancer, cervical cancer, brain cancer, lung cancer, ovarian cancer, bladder cancer, kidney cancer, liver cancer, pancreatic cancer, prostate cancer, skin cancer, tongue cancer, breast cancer, uterine cancer, stomach cancer, rectal cancer, colon cancer, blood cancer, bone cancer, oral cancer, pharynx cancer, laryngeal cancer and colon carcinoma, but the present invention is not limited thereto.

In addition, the present invention provides a method for preventing or treating cancer, including the step of administering a composition including a nanographene structure as an active ingredient to a subject in need thereof.

In addition, the present invention provides a method for supplementing anticancer therapy, including the step of administering a composition including a nanographene structure as an active ingredient to a subject in need thereof.

In addition, the present invention provides a method for preventing or ameliorating cancer, including a method of feeding (or ingesting) food including the nanographene structure as an active ingredient to a subject (by a subject) in need thereof.

In addition, the present invention provides the use of a composition including a nanographene structure as an active ingredient in the prevention or treatment of cancer.

In addition, the present invention provides the use of a composition including a nanographene structure as an active ingredient in the supplementation of anticancer therapy.

In addition, the present invention provides the use of a composition including a nanographene structure as an active ingredient in the inhibition of mitophagy.

In addition, the present invention provides the use of a composition including a nanographene structure as an active ingredient in apoptosis.

In addition, the present invention provides the use of a composition including a nanographene structure as an active ingredient in the preparation of a prophylactic or therapeutic agent for cancer.

In addition, the present invention provides the use of a composition including a nanographene structure as an active ingredient in the preparation of an anticancer supplement.

In addition, the present invention provides the use of a composition including a nanographene structure as an active ingredient in the production of food for preventing or ameliorating cancer.

In addition, the present invention provides the use of a composition including a nanographene structure as an active ingredient in the preparation of a mitophagy inhibitor.

In addition, the present invention provides the use of a composition including a nanographene structure as an active ingredient in the preparation of an apoptosis agent.

### Advantageous Effects

The composition for preventing and treating cancer through mitochondrial metabolism regulation, including a nanographene structure, has been confirmed that a specific inhibitory activity on the NIX-dependent pathway among the mitophagy mechanisms in cancer cells inhibits mitochondrial degradation, and thus, damaged mitochondria accumulate in the cancer cells, and accordingly, apoptosis of the cancer cells is induced as a result. Therefore, since the composition according to the present invention exhibits excellent anticancer effects that can be applied to various diseases related to the accumulation of abnormal cells, without causing resistance or toxicity, it can be advantageously used as a cancer prevention or treatment agent or an anticancer treatment adjuvant.

### DESCRIPTION OF DRAWINGS

FIG. 1a is a set of photographs showing the TEM analysis results for the morphologies of danGO-B and danGO-P.

FIG. 1b is a graph showing the average size and photographs showing the results of TEM analysis of NGO, NGO-NH 2 and NGO-PEG.

FIG. 2 is a graph showing the CPS analysis results for the sizes of danGO-B and danGO-P particles.

FIG. 3 is a graph showing the Raman shifts of danGO-B and danGO-P as a result of Raman spectroscopic analysis.

FIG. 4a is a graph showing peaks that are specific to active groups and PEG of danGO-B and danGO-P as a result of Raman spectroscopic analysis.

FIG. 4b is a graph showing the results of confirming the type of functional groups and functional group bonding through FT-IR of NGO-NH₂ and NGO-PEG.

FIGS. 5a and 5b are graphs showing the results of zeta potential analysis to confirm the dispersion states of danGO-B and danGO-P.

FIG. 6a is a set of graphs showing cell viability when colon cancer cell lines were treated with danGO-B and danGO-P at different concentrations.

FIG. 6b is a set of graphs showing cell viability when NGO and NGO-NH₂ were treated with colon cancer cell lines.

FIGS. 7a and 7b are photographs and graphs showing the results of flow cytometry showing the degree of cancer cell death after treatment of colon cancer cell lines with danGO-B and danGO-P.

FIG. 7c is a set of graphs showing the degree of cancer cell death after treatment of cancer cell lines with various concentrations of NGO, NGO-NH₂ and NGO-PEG.

FIGS. 8a and 8b are photographs and graphs showing immunostaining results showing the amount and rate of influx into cells as a result of treatment of colon cancer cell lines with danGO-B and danGO-P, respectively.

FIG. 9 is a set of photographs and a set of graphs showing the growth inhibitory effect of cancer cells when danGO-B and danGO-P were treated for a long period of time.

FIG. 10a is a set of graphs confirming whether the number of mitochondria in cancer cell lines increased when danGO-B and danGO-P were treated.

FIG. 10b is a set of graphs showing the results of quantitative analysis using Mitotracker on the effect of increasing mitochondria in cancer cells when cancer cells were treated with NGO, NGO-NH₂ and danGO-P at various concentrations.

FIG. 11 is a graph showing an increase in mitochondrial damage in cancer cell lines when danGO-B and danGO-P were treated as a result of analyzing changes in mitochondrial membrane potential.

FIG. 12 is a graph showing an increase in mitochondrial damage in cancer cell lines when danGO-B and danGO-P were treated as a result of analyzing changes in the ratio of mitochondrial DNA and nuclear DNA.

FIG. 13 is a set of graphs showing an increase in mitochondrial reactive oxygen species (ROS) levels in cancer cell lines when danGO-B and danGO-P were treated, as a result of flow cytometry.

FIG. 14 is a set of graphs showing an increase in total reactive oxygen species (ROS) levels in cancer cell lines when danGO-B and danGO-P were treated, as a result of DCFDA analysis.

FIG. 15 is a photograph and a graph showing the reduction of mitochondrial respiratory metabolism changes in cancer cell lines when danGO-B and danGO-P were treated.

FIG. 16 is a photograph and a graph confirming whether and how much cytochrome C was released into the cytoplasm, which is a symptom of apoptosis in cancer cell lines, when danGO-B and danGO-P were treated.

FIG. 17 is a graph showing a decrease in NIX expression in cancer cell lines when danGO-B and danGO-P were treated.

FIG. 18a is a photograph and a graph showing the involvement of danGO-B and danGO-P in the expressions of NIX, BNIP3\ and PINK1.

FIG. 18b shows the results of confirming mitophagy markers (PINK1, NIX, BNIP3) by Western blot to confirm the degree of mitochondrial autophagy of each nanographene structure.

FIGS. 19a and 19b are photographs and graphs showing the reduction of NIX expression and co-localization of graphene particles and NIX in cancer cell lines when nanographene oxide danGO-B and danGO-P were treated, as a result of fluorescence immunostaining.

FIG. 20 is a diagram showing the outline of the NIX-dependent and PINK1-dependent pathways of mitophagy.

FIG. 21a is a Western blot result confirming the autophagy flow of cancer cells when danGO-B and danGO-P were treated.

FIG. 21b is a set of Western block results confirming the autophagy flow of cancer cells when NGO, NGO-NH₂ and NGO-PEG were treated at various concentrations.

FIG. 21c shows the results of confirming the expression of proteins involved in autophagy by Western blotting after treatment of each colon cancer cell line with various concentrations of NGO-PEG, and then changing whether the drug bafilomycin was treated.

FIG. 22a is a set of photographs and a set of graphs showing whether and how much the length of the colon was preserved when danGO-B and danGO-P were administered to a mouse model in which colon cancer was induced.

FIG. 22b is a set of photographs and a set of graphs showing whether and how long the length of the colon was preserved when NGO and NGO-NH₂ were administered to a mouse model in which colon cancer was induced.

FIG. 23a is a set of photographs and a graph showing the size and number of solid tumors that were present in the lumen of the colon after administration of danGO-B and danGO-P to a mouse model in which colon cancer was induced.

FIG. 23b is a set of photographs and a graph showing the size and number of solid tumors that were present in the lumen of the colon after administration of NGO and NGO-NH 2 to a mouse model in which colon cancer was induced.

FIG. 24 shows the results of confirming the degree of inflammation occurring throughout the body of a test subject through the size and weight of the spleen when NGO and NGO-NH 2 were orally administered.

### BEST MODE

Unlike previous studies in which nanographene directly damages mitochondria in cancer cells and causes cancer cells to be killed through the generation of ROS, the present invention generates an anticancer effect by suppressing the apoptosis of cancer cells. Such an invention is based on the fact that when cancer cells are treated with nanographene oxide structures, the NIX-dependent pathway is inhibited among mitochondrial mitophagy, resulting in accumulation of damaged mitochondria and induction of the apoptosis of cancer cells, and it can be advantageously utilized for diseases caused by the overaccumulation of cells.

Hereinafter, the present invention will be described in detail.

The present invention provides a pharmaceutical composition for preventing or treating cancer, a pharmaceutical composition for supplementing anticancer therapy, a food composition for preventing or ameliorating cancer, a composition for inhibiting mitophagy and a composition for apoptosis, including a nanographene structure as an active ingredient.

In an exemplary embodiment of the present invention, the nanographene structure may be any one selected from the group consisting of a nanographene oxide, a nanographene oxide variant and graphene quantum dots, but the present invention is not limited thereto.

In an exemplary embodiment of the present invention, the nanographene oxide may exhibit any one or more of the following characteristics, but the present invention is not limited thereto:
1) it has a circular plate shape;
2) as a result of Raman spectroscopy analysis, it has a carbon-derived material-specific Raman shift; and
3) as a result of Raman spectroscopy analysis, graphene active groups —OH, —C—H and C═O are confirmed.

In an exemplary embodiment of the present invention, the nanographene oxide variant may be any one of a PEGylated nanographene oxide or an aminated nanographene oxide in which PEG (polyethylene glycol) or NH₂ (amino group) is bonded to a nanographene oxide, but the present invention is not limited thereto

In an exemplary embodiment of the present invention, the PEGylated nanographene oxide may exhibit any one or more of the following characteristics, but the present invention is not limited thereto:
1) the zeta potential of graphene ranges from −25.0 to −21.0 mV; and
2) it has a particle size of approximately 10 nm.

In an exemplary embodiment of the present invention, the aminated nanographene oxide may exhibit the following characteristic, but the present invention is not limited thereto:
1) it has a diameter of 24.4 to 38.8 nm.

As used herein, the term “nanographene oxide structure” may include nano-sized graphene oxide, nanographene oxide variants and graphene quantum dots (GQDs), but the present invention is not limited thereto.

As used herein, the term “graphene” means that a plurality of carbon atoms are covalently bonded to each other to form a polycyclic aromatic molecule, and the covalently-bonded carbon atoms form a 6-membered ring as a basic repeating unit, but it is also possible to further include a 5- or 7-membered ring.

As used herein, the term “graphene quantum dots (GQDs)” refer to graphene particles having a width, length and height of several to several tens of nm, which are prepared through appropriate processing, and the graphene quantum dots may be obtained by thermally-oxidative cutting carbon fibers, but the preparation method is not limited thereto.

The “nanographene oxide (NGO)” refers to small-sized (nano-size) nanographene oxide in which a functional group including oxygen is bonded to graphene, and it is denoted as NGO in the present invention, and it may be interchangeably used with danGO.

As used herein, the term “nanographene oxide variant” or “modified nanographene oxide” refers to a nano-sized graphene oxide in the form of substituting specific amino acids or combining amino groups (amine groups), polyethylene glycol or derivatives thereof with nano-sized graphene oxide. The nano graphene oxide variant may include a PEGylated nano graphene oxide, an aminated nano graphene oxide or a biotinylated nano graphene oxide, but the present invention is not limited thereto.

In addition, the modified nanographene oxide of the present invention includes one that is prepared by combining two or more target materials with nanographene oxide, and for example, it may be daNGO-NH₂-PEG, daNGO-PEG-NH₂ or daNGO-PEG-alpha/beta, but the present invention is not limited thereto.

The “aminated nanographene oxide (nano graphene oxide with NH₂; NGO-NH₂)” refers to a nanographene oxide in which NH₂ (amine group) is bonded to the surface of a nanographene oxide.

The “PEGylated nanographene oxide (danGO-PEG, danGO-P)” refers to a nanographene oxide in which PEG (polyethylene glycol) is bonded to the surface of a nanographene oxide.

The “biotinylated nanographene oxide (nanographene oxide with Biotin2; danGO-B)” refers to a nanographene oxide in which biotin is bonded to the surface of a nanographene oxide.

In the present invention, as the size of the nanographene oxide decreases, the surface area increases, and the number of positions where the amine group or polyethylene glycol group can be attached increases. Therefore, more amine groups or polyethylene glycol groups may be combined with a nanographene oxide.

In the present invention, the “amination” may be performed through a combination of NH₂ and a functional group (—COO—, —CO—, —OH) that is present in nano-sized graphene oxide. For this, ammonia water or ether may be used, and in an exemplary embodiment of the present invention, amination was performed through the reaction of nanographene oxide and ammonia water.

In the present invention, the “PEGylation” may be achieved through a combination of PEG and a functional group (—COO—, —CO—, —OH) that is present in nano-sized graphene oxide. In addition, it may mean that a polyethylene glycol (PEG) derivative having a structural formula represented by CH₃O—(CH₂CH₂O)ₙ (n is an integer of 2 to 4,000) is conjugated to nano-sized graphene oxide. The polyethylene glycol derivative includes various terminal groups for PEGylation, and accordingly, the polyethylene glycol derivative may be covalently bonded to the amine group, carboxyl group or Cys residue of a nano-sized graphene oxide variant through carbonyl, amide, urethane, secondary amine, thioether, disulfide and hydrazone. The PEGylation may be confirmed through PEG-specific peaks when it is analyzed by FT-IR (Fourier Transform Infrared Spectroscopy).

In the present invention, PEG (polyethyleneglycol) or a derivative thereof may be used linear or branched PEG, and for example, it may include methoxyPEG succinimidyl propionate (mPEG-SPA), methoxyPEG N-hydroxysuccinimide (mPEG-NHS), methoxyPEG aldehyde (mPEG-ALD), methoxyPEG maleimide (mPEG-MAL), multi-branched PEG and the like, but the present invention is not limited thereto.

In the present invention, the multi-branched PEG may have 2 to 8 polymer arms, but the present invention is not limited thereto. In an exemplary embodiment of the present invention, NGO-PEG was prepared with 6 arm PEG.

In the present invention, nanographene oxide refers to a material that is prepared in the form of particles having a nanometer-level size by applying various treatments to graphene oxide, and for example, the graphene may have a diameter of 1 to 50 nm, 1 to 40 nm, 1 to 30 nm, 1 to 25 nm, 1 to 20 nm, 1 to 15 nm, 1 to 12 nm, 3 to 50 nm, 3 to 40 nm, 3 to 30 nm, 3 to 25 nm, 3 to 20 nm, 3 to 15 nm or 3 to 12 nm, but the present invention is not limited thereto. The PEGylated nanographene oxide according to an exemplary embodiment of the present invention may exhibit a diameter in the range of about 1 to 25 nm, and form a peak at 10 nm to 13 nm to have an average diameter of about 10 nm. In addition, the biotinylated nanographene oxide according to an exemplary embodiment of the present invention may exhibit a diameter in the range of about 1 to 50 nm, and form a peak at 10 nm to 13 nm to have an average diameter of about 10 nm.

In the present invention, the diameter may mean D50, but the present invention is not limited thereto.

In the present invention, D50 means the particle size at a point where the cumulative curve becomes 50% when the cumulative curve of the particle size distribution is obtained with the total weight as 100%.

For example, the NGO-NH₂ may have a diameter of 5 to 80 nm, 5 to 70 nm, 5 to 60 nm, 5 to 50 nm, 5 to 43 nm, 10 to 80 nm, 10 to 70 nm, 10 to 60 nm, 10 to 50 nm, 10 to 43 nm, 20 to 80 nm, 20 to 70 nm, 20 to 60 nm, 20 to 50 nm, or 20 to 43 nm, but the present invention is not limited thereto. In an exemplary embodiment of the present invention, NGO-NH₂ having a diameter of about 24.4 to 38.8 nm was synthesized.

For example, the NGO-PEG may have a diameter of 5 to 90 nm, 5 to 80 nm, 5 to 70 nm, 5 to 60 nm, 5 to 51 nm, 10 to 90 nm, 10 to 80 nm, 10 to 70 nm, 10 to 60 nm, 10 to 51 nm, 20 to 90 nm, 20 to 80 nm, 20 to 70 nm, 20 to 60 nm, or 20 to 51 nm, but the present invention is not limited thereto. In an exemplary embodiment of the present invention, NGO-PEG having a diameter of about 25.0 to 45.4 nm was synthesized.

In the present invention, the zeta potential of nanographene oxide may be −60 to 0 mV, −60 to −5 mV, −60 to −10 mV, −60 to −15 mV, −60 to −21 mV, −60 to −25 mV, −60 to −30 mV, −60 to −35 mV, −50 to 0 mV, −45 to 0 mV, −40 to 0 mV, −35 to 0 mV, −30 to 0 mV, −25 to 0 mV, −21 to 0 mV, −50 to −5 mV, −50 to −10 mV, −50 to −15 mV, −50 to −21 mV, −45 to −5 mV, −45 to −10 mV, −45 to −15 mV, −45 to −21 mV, −40 to −5 mV, −40 to −10 mV, −40 to −15 mV, −40 to −21 mV, −35 to −5 mV, −35 to −10 mV, −35 to −15 mV, or −35 to −21 mV, but the present invention is not limited thereto. The PEGylated nanographene oxide according to an exemplary embodiment of the present invention may exhibit a zeta potential change in the range of about −40 to 0 mV, and exhibit a peak at about −21 mV, thereby indicating a good dispersion state. In addition, the biotinylated nanographene oxide may exhibit a zeta potential change in the range of about −50 to 0 mV, and exhibit a peak at about −34.5 mV, thereby indicating a good dispersion state.

In an exemplary embodiment of the present invention, the structure may inhibit mitochondrial mitophagy, but the present invention is not limited thereto.

In the present invention, mitophagy may refer to an intracellular degradation mechanism (autophagy) that occurs within mitochondria to remove damaged or unnecessary mitochondria, and the nanographene structure according to the present invention may inhibit mitophagy.

The “inhibition” may mean both of inhibiting the start of a mechanism that induces mitochondrial mitophagy or stopping a mechanism that has already started. For example, the structure of the present invention may inhibit mitophagy by interfering with the action of substances involved in any one or more of the downstream of the mitophagy mechanism, such as enzymes and proteins.

In a specific embodiment of the present invention, the structure may specifically inhibit NIX, but the present invention is not limited thereto.

In the present invention, “NIX” is a protein that is associated with the NIX-dependent pathway among mitophagy mechanisms generated in damaged mitochondria, and the nanographene structure of the present invention may specifically inhibit the activity of NIX.

The “inhibition” may mean inhibiting or reducing the expression of NIX, or attenuating the activity of NIX that is already expressed.

The “specific” may mean that there is no inhibitory effect on BNIP3 and PINK1 that are associated with the BNIP3-dependent pathway and PINK1-dependent pathway, which are other mechanisms of autophagy occurring in damaged mitochondria.

In a specific embodiment of the present invention, the structure may be characterized in that it does not inhibit BNIP3 and PINK1, but the present invention is not limited thereto.

In the present invention, “not inhibiting BNIP3 and PINK1” may mean not inhibiting the expression of the target substance itself or not reducing the expression level below a normal level. Therefore, the above expression means that when the composition of the present invention is treated, BNIP3 and PINK1 may be expressed at a level similar to or higher than a common level in the art, and in this case, the common level is a statistically insignificantly low level.

In an exemplary embodiment of the present invention, the structure may be included at a concentration of 0.01 to 200 μg/mL, but the present invention is not limited thereto.

In addition, the nanographene oxide may be included at a concentration of 0.01 to 200 μg/mL, 0.01 to 100 μg/mL, 0.01 to 50 μg/mL, 0.01 to 45 μg/mL, 0.01 to 40 μg/mL, 0.01 to 35 μg/mL, 0.01 to 30 μg/mL, 0.01 to 25 μg/mL, 0.01 to 20 μg/mL, 0.1 to 200 μg/mL, 0.1 to 200 μg/mL, 0.1 to 150 μg/mL, 0.1 to 100 μg/mL, 0.1 to 200 μg/mL 50 μg/mL, 0.1 to 45 μg/mL, 0.1 to 40 μg/mL, 0.1 to 35 μg/mL, 0.1 to 30 μg/mL, 0.1 to 25 μg/mL, 0.1 to 20 μg/mL, 0.5 to 200 μg/mL, 0.5 to 150 μg/mL, 0.5 to 100 μg/mL, 0.5 to 50 μg/mL, 0.5 to 45 μg/mL, 0.5 to 40 μg/mL, 0.5 to 35 μg/mL, 0.5 to 30 μg/mL, 0.5 to 25 μg/mL, 0.5 to 20 μg/mL, 1 to 200 μg/mL, 1 to 150 μg/mL, 1 to 100 μg/mL, 1 to 50 μg/mL, 1 to 45 μg/mL, 1 to 40 μg/mL, 1 to 35 μg/mL, 1 to 30 μg/mL, 1 to 25 μg/mL, 1 to 20 μg/mL, 2 to 200 μg/mL, 2 to 150 μg/mL, 2 to 100 μg/mL, 2 to 50 μg/mL, 2 to 45 μg/mL, 2 to 40 μg/mL, 2 to 35 μg/mL, 2 to 30 μg/mL, 2 to 25 μg/mL, 2 to 20 μg/mL, 5 to 200 μg/mL, 5 to 150 μg/mL, 5 to 100 μg/mL, 5 to 50 μg/mL, 5 to 45 μg/mL, 5 to 40 μg/mL, 5 to 35 μg/mL, 5 to 30 μg/mL, 5 to 25 μg/mL, 5 to 20 μg/mL, 10 to 200 μg/mL, 10 to 150 μg/mL, 10 to 100 μg/mL, 10 to 50 μg/mL, 10 to 45 μg/mL, 10 to 40 μg/mL, 10 to 35 μg/mL, 10 to 30 μg/mL, 10 to 25 μg/mL, 10 to 20 μg/mL, 15 to 200 μg/mL, 15 to 150 μg/mL, 15 to 100 μg/mL, 15 to 50 μg/mL, 15 to 45 μg/mL, 15 to 40 μg/mL, 15 to 35 μg/mL, 15 to 30 μg/mL, 15 to 25 μg/mL, 15 to 20 μg/mL, 20 to 200 μg/mL, 20 to 150 μg/mL, 20 to 100 μg/mL, 20 to 50 μg/mL, 20 to 30 μg/mL, or 20 to 25 μg/mL, but the present invention is not limited thereto.

In addition, the NGO-NH₂ may be included at a concentration of 0.01 to 200 μg/mL, 0.01 to 100 μg/mL, 0.01 to 50 μg/mL, 0.01 to 45 μg/mL, 0.01 to 40 μg/mL, 0.01 to 35 μg/mL, 0.01 to 30 μg/mL, 0.01 to 25 μg/mL, 0.01 to 20 μg/mL, 0.05 to 200 μg/mL, 0.05 to 100 μg/mL, 0.05 to 50 μg/mL, 0.05 to 45 μg/mL, 0.05 to 0.05 μg/mL 40 μg/mL, 0.05 to 35 μg/mL, 0.05 to 30 μg/mL, 0.05 to 25 μg/mL, 0.05 to 20 μg/mL, 0.5 to 200 μg/mL, 0.5 to 100 μg/mL, 0.5 to 25 μg/mL 50 μg/mL, 0.5 to 45 μg/mL, 0.5 to 40 μg/mL, 0.5 to 35 μg/mL, 0.5 to 30 μg/mL, 0.5 to 25 μg/mL, 0.5 to 20 μg/mL, 1 to 200 μg/mL, 1 to 100 μg/mL, 1 to 50 μg/mL, 1 to 45 μg/mL, 1 to 40 μg/mL, 1 to 35 μg/mL, 1 to 30 μg/mL, 1 to 25 μg/mL, 1 to 20 μg/mL, 2 to 200 μg/mL, 2 to 100 μg/mL, 2 to 50 μg/mL, 2 to 45 μg/mL, 2 to 40 μg/mL, 2 to 35 μg/mL, 2 to 30 μg/mL, 2 to 25 μg/mL, 2 to 20 μg/mL, 4 to 200 μg/mL, 4 to 100 μg/mL, 4 to 50 μg/mL, 4 to 45 μg/mL, 4 to 40 μg/mL, 4 to 35 μg/mL, 4 to 30 μg/mL, 4 to 25 μg/mL, 4 to 20 μg/mL, 5 to 200 μg/mL, 5 to 100 μg/mL, 5 to 50 μg/mL, 5 to 45 μg/mL, 5 to 40 μg/mL, 5 to 35 μg/mL, 5 to 30 μg/mL, 5 to 25 μg/mL, 5 to 20 μg/mL, 10 to 200 μg/mL, 10 to 100 μg/mL, 10 to 50 μg/mL, 10 to 45 μg/mL, 10 to 40 μg/mL, 10 to 35 μg/mL, 10 to 30 μg/mL, 10 to 25 μg/mL, 10 to 20 μg/mL, 15 to 200 μg/mL, 15 to 100 μg/mL, 15 to 50 μg/mL, 15 to 45 μg/mL, 15 to 40 μg/mL, 15 to 35 μg/mL, 15 to 30 μg/mL, 15 to 25 μg/mL, 15 to 20 μg/mL, 20 to 200 μg/mL, 20 to 100 μg/mL, 20 to 50 μg/mL, 20 to 45 μg/mL, 20 to 40 μg/mL, 20 to 35 μg/mL, 20 to 30 μg/mL, or 20 to 25 μg/mL, but the present invention is not limited thereto.

In addition, the NGO-PEG may be included at a concentration of 0.01 to 200 μg/mL, 0.01 to 100 μg/mL, 0.01 to 50 μg/mL, 0.01 to 45 μg/mL, 0.01 to 40 μg/mL, 0.01 to 35 μg/mL, 0.01 to 30 μg/mL, 0.01 to 25 μg/mL, 0.01 to 20 μg/mL, 0.1 to 200 μg/mL, 0.1 to 100 μg/mL, 0.1 to 50 μg/mL, 0.1 to 45 μg/mL, 0.1 to 40 μg/mL, 0.1 to 35 μg/mL, 0.1 to 30 μg/mL, 0.1 to 25 μg/mL, 0.1 to 20 μg/mL, 1 to 200 μg/mL, 1 to 100 μg/mL, 1 to 50 μg/mL, 1 to 45 μg/mL, 1 to 40 μg/mL, 1 to 35 μg/mL, 1 to 30 μg/mL, 1 to 25 μg/mL, 1 to 20 μg/mL, 2 to 200 μg/mL, 2 to 100 μg/mL, 2 to 50 μg/mL, 2 to 45 μg/mL, 2 to 40 μg/mL, 2 to 35 μg/mL, 2 to 30 μg/mL, 2 to 25 μg/mL, 2 to 20 μg/mL, 4 to 200 μg/mL, 4 to 100 μg/mL, 4 to 50 μg/mL, 4 to 45 μg/mL, 4 to 40 μg/mL, 4 to 35 μg/mL, 4 to 45 μg/mL 30 μg/mL, 4 to 25 μg/mL, 4 to 20 μg/mL, 5 to 200 μg/mL, 5 to 100 μg/mL, 5 to 50 μg/mL, 5 to 45 μg/mL, 5 to 40 μg/mL, 5 to 35 μg/mL, 5 to 30 μg/mL, 5 to 25 μg/mL, 5 to 20 μg/mL, 10 to 200 μg/mL, 10 to 100 μg/mL, 10 to 50 μg/mL, 10 to 45 μg/mL, 10 to 40 μg/mL, 10 to 35 μg/mL, 10 to 30 μg/mL, 10 to 30 μg/mL 25 μg/mL, 10 to 20 μg/mL, 15 to 200 μg/mL, 15 to 100 μg/mL, 15 to 50 μg/mL, 15 to 45 μg/mL, 15 to 40 μg/mL, 15 to 35 μg/mL, 15 to 30 μg/mL, 15 to 25 μg/mL, 15 to 20 μg/mL, 20 to 200 μg/mL, 20 to 100 μg/mL, 20 to 50 μg/mL, 20 to 45 μg/mL, 20 to 40 μg/mL, 20 to 35 μg/mL, 20 to 30 μg/mL, or 20 to 25 μg/mL, but the present invention is not limited thereto.

As used herein, the term “pharmaceutical composition for supplementing anticancer therapy” may mean generating or enhancing an anticancer effect when it is co-administered with an anticancer agent, but the present invention is not limited thereto.

In addition, the “anticancer effect” may mean generating or enhancing the effect of an anticancer drug itself, or generating or enhancing the reactivity to the anticancer drug, and it may mean generating or enhancing the effect of combination treatment with other anticancer drugs or anticancer treatment methods, but the present invention is not limited thereto.

The pharmaceutical composition for supplementing anticancer therapy according to the present invention may be administered in combination with an anticancer agent, and the combination period, cycle, dose, concentration, method and the like may be administered in combination in the most preferred way in consideration of circumstances such as the clinical condition of a patient, the type of cancer, the type of anticancer agent used in combination, the type of other compositions and the like.

In addition, the pharmaceutical composition for supplementing anticancer therapy may be prepared with other materials that can be generally added to enhance the adjuvant effect of an anticancer agent, and it may be administered in combination with other compositions.

In addition, the pre-treatment or post-treatment method, storage method and usage method that are generally performed to enhance the auxiliary effect may be applied without limitation.

In a specific embodiment of the present invention, the cancer may be one or more selected from the group consisting of thyroid cancer, cervical cancer, brain cancer, lung cancer, ovarian cancer, bladder cancer, kidney cancer, liver cancer, pancreatic cancer, prostate cancer, skin cancer, tongue cancer, breast cancer, uterine cancer, stomach cancer, rectal cancer, colon cancer, blood cancer, bone cancer, oral cancer, pharynx cancer, laryngeal cancer and colon carcinoma, but the present invention is not limited thereto.

As used herein, the term “cancer” refers to a generic term for diseases caused by cells having aggressive properties in which cells divide and grow in defiance of normal growth limits, invasive properties in which cells invade surrounding tissue, and metastatic properties in which cells spread to other parts of the body. In addition, cancer may mean cancer in a general sense, for example, benign and malignant tumors, and may encompass cancers at all stages.

The “colon cancer” refers to malignant tumors composed of cancer cells in the colon, most of which are adenocarcinomas occurring in the mucous membrane of the colon, and other than the above, there are lymphoma, sarcoma, squamous cell carcinoma and metastatic lesions of other cancers. The colon cancer may be divided into primary colon cancer that occurs in the large intestine and metastatic colon cancer that has spread to the large intestine from other organs based on metastasis.

As used herein, the term “autophagy” refers to lysosome-mediated catabolic activity that maintains cell homeostasis through the degradation and reuse of cytoplasmic components and organelles.

As used herein, the term “mitophagy” is mitochondria-specific autophagy that removes mitochondria in order to maintain mitochondrial properties (L. Liu et al., Cell Res., 24:787-795, 2014). It is known that mitophagy prevents mitochondrial dysfunction, inflammation, apoptosis and severe oxidative stress, which are closely associated with the pathological progression of neurological and metabolic diseases (D. C. Wallace et al., Annu. Rev. Genet., 39: 359-407, 2005).

The composition of the present invention may be used to kill unnecessary cells due to aging or damage by inhibiting mitophagy, which is an autophagy of intracellular mitochondria, and it may be used to prevent or treat various diseases caused therefrom.

For example, tumors may be suppressed by inducing apoptosis by inhibiting the mitophagy of damaged cells in tumor cells.

In addition, it may be used for various nervous system diseases caused by the accumulation of unnecessary cells, such as degenerative brain diseases, Alzheimer's disease, dementia and the like, and it may be used for autoimmune diseases, such as systemic lupus erythematosus, rheumatoid arthritis, Sjogren's syndrome, Behcet's disease, systemic sclerosis, polymyositis, dermatomyositis, rheumatoid arthritis and the like.

The composition for preventing or treating cancer according to the present invention has the same meaning as the pharmaceutical composition for preventing or treating cancer, and it may further include appropriate carriers, excipients and diluents that are commonly used in the preparation of pharmaceutical compositions. The excipient may be, for example, one or more selected from the group consisting of a diluent, a binder, a disintegrant, a lubricant, an adsorbent, a moisturizer, a film-coating material and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions or aerosols. The preparation for external use may have a formulation such as creams, gels, patches, sprays, ointments, plasters, lotions, liniments, pastes or cataplasmas.

As the carrier, the excipient and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate and mineral oil may be used.

For formulation, commonly used diluents or excipients such as fillers, thickeners, binders, wetting agents, disintegrants and surfactants are used for preparation.

As additives of tablets, powders, granules, capsules, pills and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC) 1928, HPMC 2208, HPMC 2906, HPMC 2910, propylene glycol, casein, calcium lactate and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose and sodium carboxymethylcellulose may be used.

In syrups according to the present invention, a white sugar solution, other sugars or sweeteners and the like may be used, and as necessary, a fragrance, a colorant, a preservative, a stabilizer, a suspending agent, an emulsifier, a viscous agent or the like may be used.

In emulsions according to the present invention, purified water may be used, and as necessary, an emulsifier, a preservative, a stabilizer, a fragrance or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC) 1828, HPMC 2906, HPMC 2910 and the like may be used, and as necessary, a surfactant, a preservative, a stabilizer, a colorant and a fragrance may be used.

Injections according to the present invention may include solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂) and ethylenediamine tetraacetic acid; sulfating agents such as 0.1% sodium bisulfide, sodium formaldehyde sulfoxylate, thiourea, disodium ethylenediaminetetraacetate and acetone sodium bisulfite; a pain relief agent such as benzyl alcohol, chlorobutanol, procaine hydrochloride, glucose and calcium gluconate; and suspending agents such as sodium CMC, sodium alginate, Tween 80 and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan(propylene glycol monostearate), glycerin, Adeps solidus, buytyrum Tego-G, cebes Pharma 16, hexalide base 95, cotomar, Hydrokote SP, S-70-XXA, S-70-XX75(S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro (OSI, OSIX, A, B, C, D, H, L), suppository base IV types (AB, B, A, BC, BBG, E, BGF, C, D, 299), suppostal N, Es, Wecoby (W, R, S, M, Fs) and tegester triglyceride matter (TG-95, MA, 57) may be used.

Solid preparations for oral administration include tablets, pills, powders, granules, capsules and the like, and such solid preparations are formulated by mixing the composition with at least one excipient, for example, starch, calcium carbonate, sucrose, lactose, gelatin and the like. In addition to simple excipients, lubricants such as magnesium stearate and talc are also used.

Examples of liquid preparations for oral administration include suspensions, liquids for internal use, emulsions, syrups and the like, and these liquid preparations may include, in addition to simple commonly used diluents, such as water and liquid paraffin, various types of excipients, for example, a wetting agent, a sweetener, a fragrance, a preservative and the like. Preparations for parenteral administration include an aqueous sterile solution, a non-aqueous solvent, a suspension, an emulsion, a freeze-dried preparation and a suppository. Non-limiting examples of the non-aqueous solvent and the suspension include propylene glycol, polyethylene glycol, a vegetable oil such as olive oil and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The pharmaceutical composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art to which the present invention pertains.

The pharmaceutical composition of the present invention may be administered to a subject via various routes. All administration methods can be predicted, and the pharmaceutical composition may be administered via, for example, oral administration, subcutaneous injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration or the like.

The pharmaceutical composition of the present invention is determined depending on the type of a drug, which is an active ingredient, along with various related factors such as a disease to be treated, administration route, the age, gender, and body weight of a patient and the severity of diseases.

As used herein, the term “subject” refers to a subject in need of treatment of a disease, and more specifically refers to a mammal such as a human or a non-human primate, a mouse, a rat, a dog, a cat, a horse and a cow.

As used herein, the term “administration” refers to providing a subject with a predetermined composition of the present invention by using an arbitrary appropriate method.

As used herein, the term “prevention” means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “amelioration” as used herein means all actions that reduce the degree of parameters associated with a target disease, for examples, symptoms via administration of the composition according to the present invention.

In the present invention, when it is said to “include” a certain component, it means that it may further include other components unless otherwise stated.

In addition, the present invention relates to a food composition for preventing or improving cancer, including a nanographene structure as an active ingredient.

Food in the present invention may mean including all types of food, such as health functional food, functional food and general food.

When the nanographene structure of the present invention is used as a food additive, the nanographene structure may be added as it is or used together with other foods or food ingredients, and it may be appropriately used according to the conventional method. The mixing amount of the active ingredient may be appropriately determined according to the purpose of use (prevention, health or therapeutic treatment). In general, when producing food or beverage, the nanographene structure of the present invention may be added in an amount of 15 wt. % or less or 10 wt. % or less based on the raw material. However, in the case of long-term intake for the purpose of health and hygiene or health control, the amount may be less than the above range, and since there is no problem in terms of safety, the active ingredient may be used in an amount that is greater than the above range.

There is no particular limitation on the type of food. Examples of food to which the above substances can be added include meat, sausages, bread, chocolates, candies, snacks, confectionery, pizza, ramen, other noodles, gums, dairy products including ice creams, various soups, beverages, tea, drinks, alcoholic beverages, vitamin complexes and the like, and they include all health functional foods in the conventional sense.

The health beverage composition according to the present invention may contain various flavoring agents or natural carbohydrates as additional components, like conventional beverages. The above-described natural carbohydrates are monosaccharides such as glucose and fructose, disaccharides such as maltose and sucrose, polysaccharides such as dextrins and cyclodextrins, and sugar alcohols such as xylitol, sorbitol and erythritol. As the sweetener, natural sweeteners such as thaumatin and stevia extract, or synthetic sweeteners such as saccharin and aspartame may be used. The proportion of the natural carbohydrate is generally about 0.01 to 0.20 g, or about 0.04 to 0.10 g per 100 mL of the composition of the present invention.

In addition to the above, the composition of the present invention may contain various nutrients, vitamins, electrolytes, flavors, colorants, pectic acid and salts thereof, alginic acid and salts thereof, organic acids, protective colloidal thickeners, pH adjusters, stabilizers, preservatives, glycerin, alcohols, carbonation agents used in carbonated beverages and the like. In addition, the composition of the present invention may contain fruit flesh for preparing natural fruit juice, fruit juice beverages and vegetable beverages. These components may be used independently or in combination. The ratio of these additives is not critically important, but is generally selected in the range of 0.01 to 0.20 parts by weight per 100 parts by weight of the composition of the present invention.

The terms used in the present invention have been selected from general terms that are currently widely used as much as possible while considering the functions in the present invention, but these may vary depending on the intention of a person skilled in the art or precedent, the emergence of new technologies and the like. Additionally, in a specific case, there may also be terms that are arbitrarily selected by the Applicant, and in this case, the meanings will be described in detail in the Description of the Invention that corresponds thereto. Therefore, the terms used in the present invention must be defined based on the meanings of the terms and the overall content of the present invention, not simply by the names of the terms.

### MODES OF THE INVENTION

Hereinafter, a preferred exemplary embodiment is presented to aid understanding of the present invention. However, the following examples are provided to more easily understand the present invention, and the content of the present invention is not limited by the following examples.

### EXAMPLE

### Example 1. Fabrication of Nanographene Structure

1-1. Fabrication of Nanographene Oxide (Deka-Nano Sized Graphene Oxide; danGO or NGO)

Nano-sized graphene oxide was synthesized through Taylor's method. In order to manufacture nano-sized GO, the obtained GO was subjected to nanoization through various treatment methods such as separate strong physical impact or ultrasonic waves, and the treated nanographene oxide was vacuum-filtered with a cellulose nitrate membrane filter (0.22 μm, GE Healthcare).

### 1-2. Synthesis of Modified Nanographene Oxide

After fabricating the nanographene oxide structure by the NGO fabrication method of 1-1, an amine group (—NH₂), biotin (-Biotin) or polyethylene glycol (-PEG) functional group was additionally bonded to synthesize a modified nanographene oxide, thereby fabricating NGO-NH₂, danGO-B and danGO-PEG (danGO-P), respectively. In this case, danGO-B was fabricated as a control group.

Specifically, after preparing the nanographene oxide structure of Example 1-1, an amine group (—NH₂) functional group was additionally bonded to synthesize NGO-NH₂. The bond between the nanographene oxide and the amine group was achieved through a reaction between the nanographene oxide and ammonia water. The concentration of ammonia water can be applied from 10 to 40%, and 1 g of nanographene oxide was reacted based on 100 mL of ammonia water. Thereafter, various types of energy (heat, pressure, etc.) were applied to facilitate the reaction.

Additionally, in order to bind biotin to the nanographene oxide structure, 0.1 to 5 wt. % of nanographene oxide was mixed with EDC (1-Ethyl-3-(3-dimethylaminopropyl)carbodiimide) (22981, Thermofisher Scientific) at a ratio of 5:1. NHS (N-Hydroxysulfosuccinimide) (24525, Thermofisher Scientific) was introduced to this solution at a level of 50% of the injected EDC, stirred for 1 hour or more, and then titrated to pH 4.5. Thereafter, a Biotin solution (14400, Sigma-Aldrich) (distilled water:Biotin=1:0.5 to 1:3 input) was introduced and reacted for 1 hour or more, and then, the material floating in the upper layer was collected through a centrifugal process.

In order to bind PEG to the nanographene oxide structure, PEG (22105, Thermofisher Scientific) was dissolved in distilled water and mixed with 0.01 to 1 wt. % of nanographene oxide at a ratio of 1:2 to 1:20. TEA (Triethyl amine) (471283, Sigma Aldrich) was introduced to this solution (PEG:TEA=1:0.05˜ 1:0.1), and EDC and distilled water were added to this solution, and after stirring for 12 hours or more (PEG:EDC=1:0.5 to 1:5), the solution was washed through centrifugation to remove PEG that did not react with graphene.

### Example 2. Verification of Properties of Nanographene Structure

TEM (Transmission electron microscopy) analysis, CPS analysis, Raman spectroscopy and Zeta potential analysis were performed to verify the morphological and chemical properties of each modified nanographene structure prepared in Example 1.

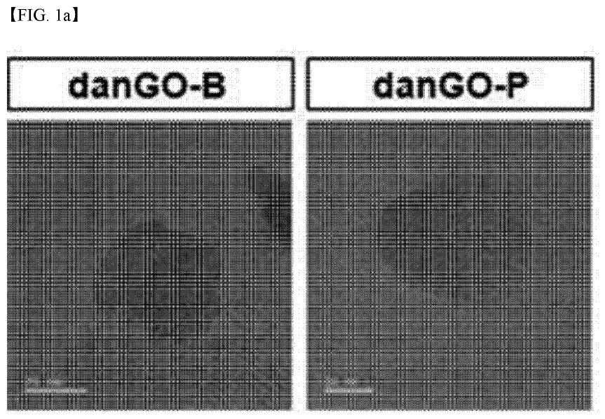

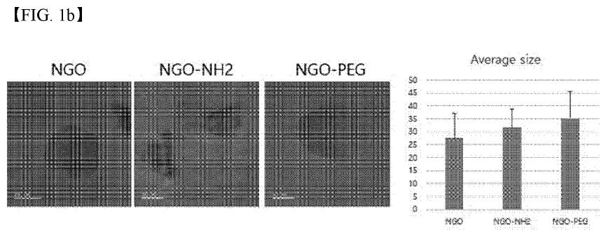

As a result of TEM analysis, it was confirmed that each graphene had a circular plate shape (FIGS. 1a and 1b), and NGO-NH₂ was measured to have a diameter of 24.4 to 38.8 nm (FIG. 1b).

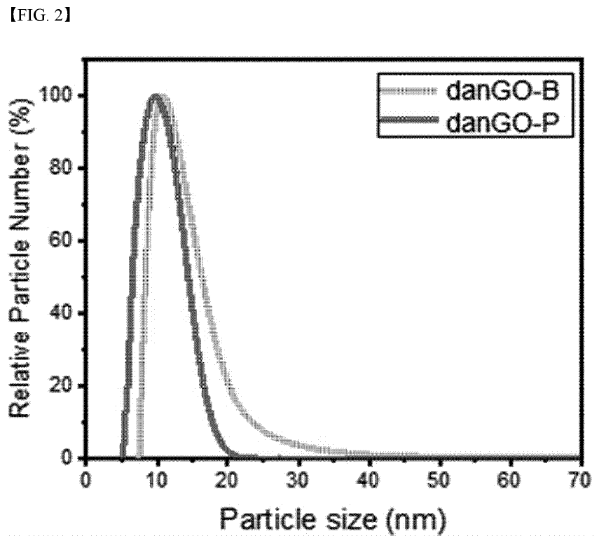

In addition, as a result of CPS analysis, graphene particle size distribution graphs showed similar patterns, and the peak of both graphs was shown to be about 10 nm, which was confirmed to be the representative particle size of each graphene (FIG. 2).

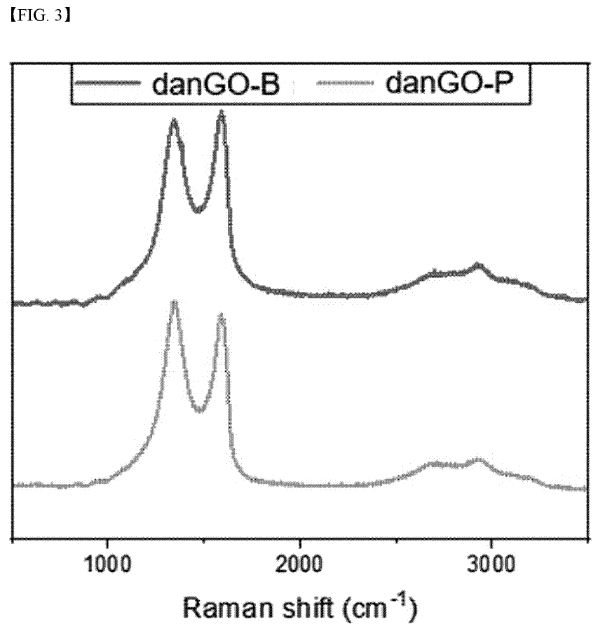

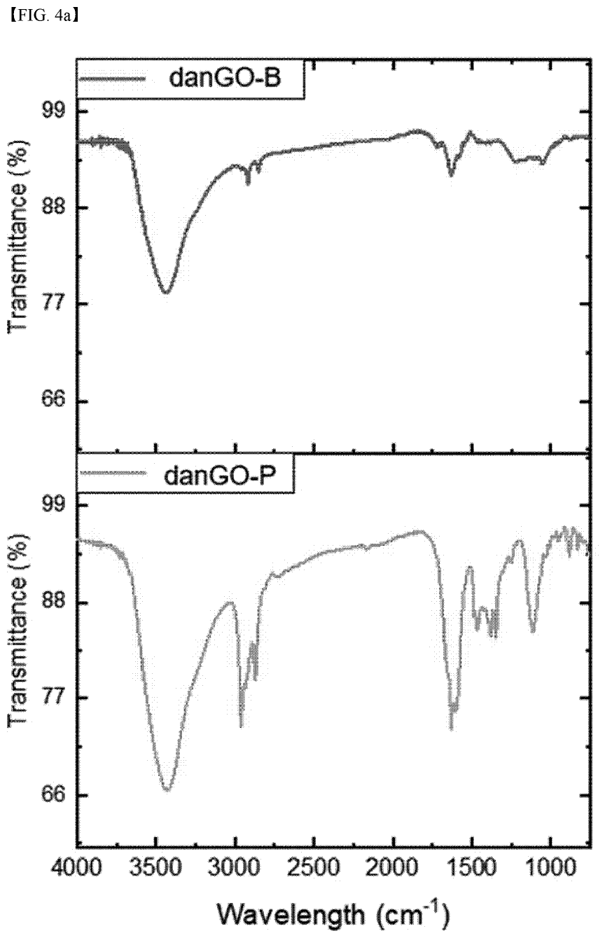

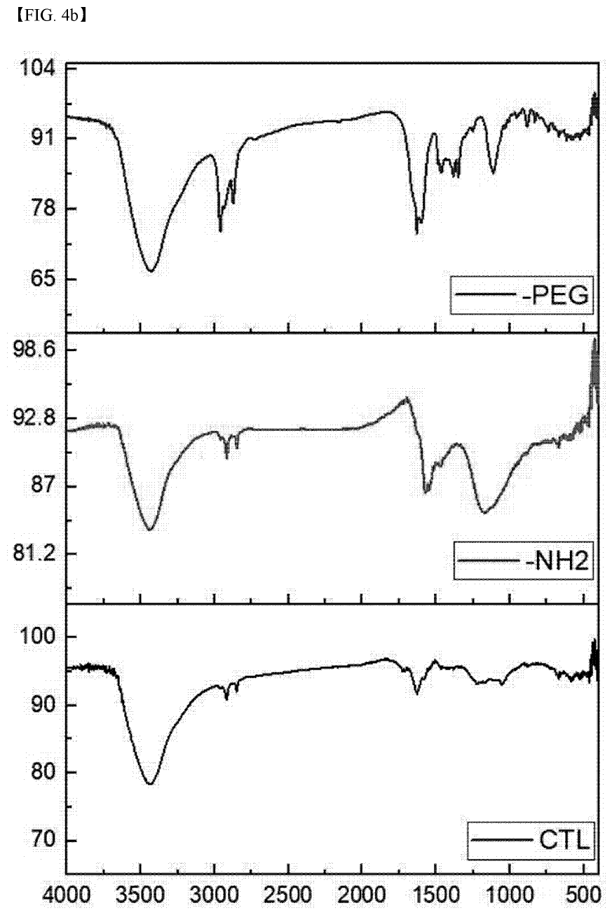

In addition, as a result of Raman spectroscopy analysis, D band (1,350 cm⁻¹) and G band (1,588 cm⁻¹) were observed in both graphenes, which confirmed that they possessed specific Raman shifts of carbon-derived materials (FIG. 3). In addition, OH (3,440 cm⁻¹), CH (2,918 cm⁻¹) and C═O (1,630 cm⁻¹), which are active groups bonded to graphene, were commonly observed, and the PEG-specific peaks were observed at 1,465 cm⁻¹, 1,349 cm⁻¹ and 1,113 cm⁻¹, and —NH₂ specific peaks were observed at 1,570 cm⁻¹ and 1,170 cm⁻¹, which indicated that functional group bonding was normally performed (FIG. 4a and FIG. 4b).

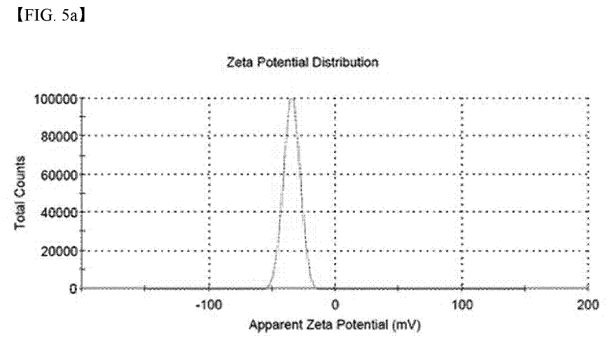

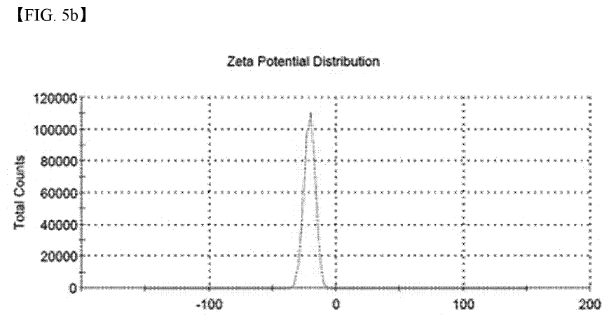

Finally, as a result of performing zeta potential analysis to confirm the dispersion state of graphene, the zeta potentials of danGO-B and danGO-P were shown at −34.5 mV and −21.0 mV, respectively, which confirmed that both groups had good dispersion stability (FIGS. 5a and 5b).

### Example 3. Confirmation of Anticancer Effect of Nanographene Oxide and Modified Structures Thereof

### Example 3-1. Confirmation of Effect of Cancer Cell Death of Nanographene Oxide and Modified Structures Thereof

In order to confirm the effect of cancer cell death of nanographene oxide and modified structures thereof, cancer cell viability and the degree of cancer cell death were analyzed. Specifically, SW480 and SW620 cell lines were respectively treated with danGO-PEG and danGO-B at different concentrations (0.1, 0.5, 2, 5, 10, 20, 50 μg/mL), and cell viability was analyzed by using MTT assay (M6494 Thermofisher Scientific).

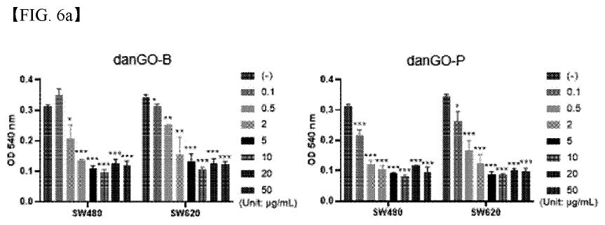

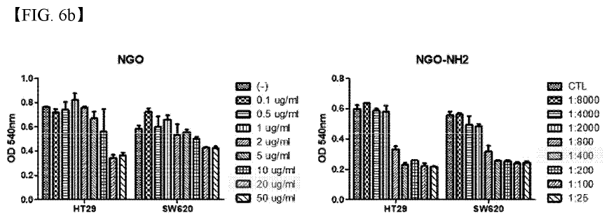

As a result, it was confirmed that each modified nanographene oxide has an effect of reducing the viability of cancer cells (FIG. 6a).

The degree of cancer cell death of danGO-PEG and danGO-B at the concentration (10 μg/mL) confirmed to significantly decrease viability was analyzed. In order to specifically analyze the degree of cancer cell death, after treating each modified nanographene oxide structure to a colon cancer cell line, the cancer cell line was treated with graphene at each concentration, and then stained with Annexin V (A23204, Invitrogen) to analyze the degree of cancer cell death by a flow cytometer.

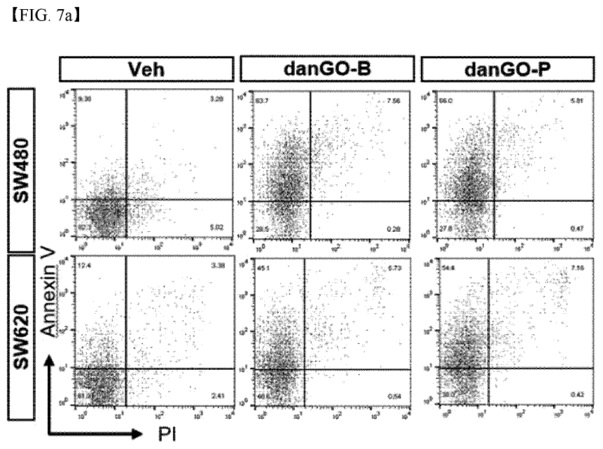

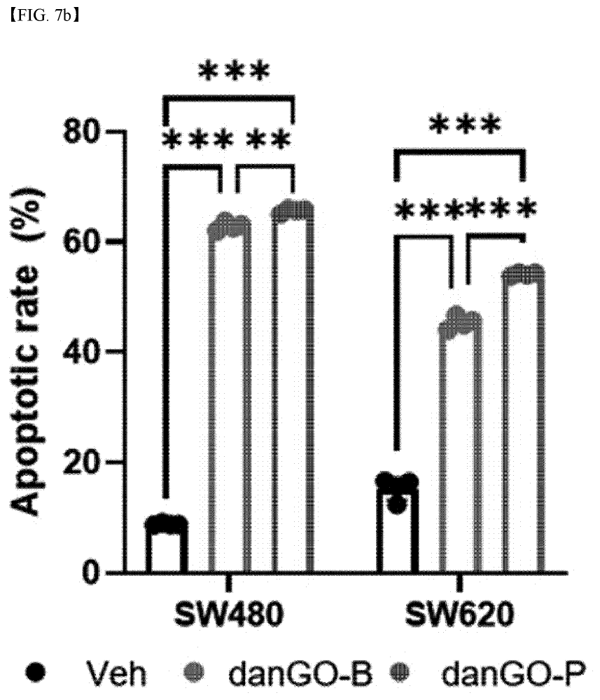

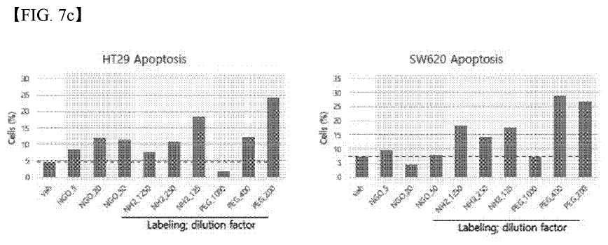

As a result, similar to the above experimental results, when all of the experimental groups and the control group were treated, the apoptosis of cancer cells was greatly increased, and particularly, the apoptosis effect of the experimental groups was confirmed to be superior to that of the control group (FIGS. 7a and 7b). Therefore, various anticancer effects were confirmed with respect to graphene at 10 μg/mL, which had the most excellent cancer cell killing effect.

For the first experiment of the two experiments, only the concentrations of danGO and NGO-NH₂ were varied (cases where danGO was 0.1, 0.5, 1, 2, 5, 10, 20 and 50 μg/mL, and NGO-NH₂ was 0.5, 1, 2, 4, 5, 10, 20 and 40 μg/mL) to perform the same experiment, and the results are shown in FIG. 6b.

In addition, for the second experiment, danGO (NGO), NGO-NH₂ and danGO-PEG were treated at 3 concentrations (NGO 5, 20 and 50 μg/mL, NGO-NH₂ 4, 20 and 40 μg/mL, NGO-PEG 5, 20 and 40 μg/mL) to perform the experiment in the same manner as above (FIG. 7c).

As a result, when NGO-NH₂ and NGO-PEG were treated, the apoptosis of cancer cells was maximized, and NGO-NH₂ exceeded a vehicle at all concentrations, but when NGO-PEG was treated at a low concentration of 5 μg/mL, it was less than or comparable to a vehicle (FIG. 7c). However, at other concentrations, a significant apoptosis increasing effect was confirmed. That is, it suggested that the modified nanographene (NGO-NH₂, NGO-PEG) increases the cancer cell killing effect.

### Example 3-2. Confirmation of the Influx Effect of Modified Nanographene Oxide into Cancer Cells

In order to quantitatively determine the influx effect and amount of modified nanographene oxide into cancer cells, 10 μg/mL of danGO-PEG and danGO-B were treated with colon cancer cell lines to perform immunostaining analysis. Specifically, colon cancer cell lines were prepared by primary colon cancer cells SW480 and metastatic colon cancer cells SW620, and the cell lines were respectively treated with an experimental group (danGO-PEG) and a control group, and the immunostaining analysis was performed on day 1, day 2 and day 3. After probing with Biotin-conjugated anti-PEG antibody (ab53449, Abcam), danGO-Biotin and -PEG were detected with FITC-conjugated streptavidin in the experimental group.

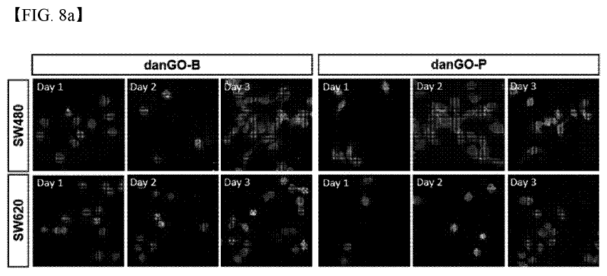

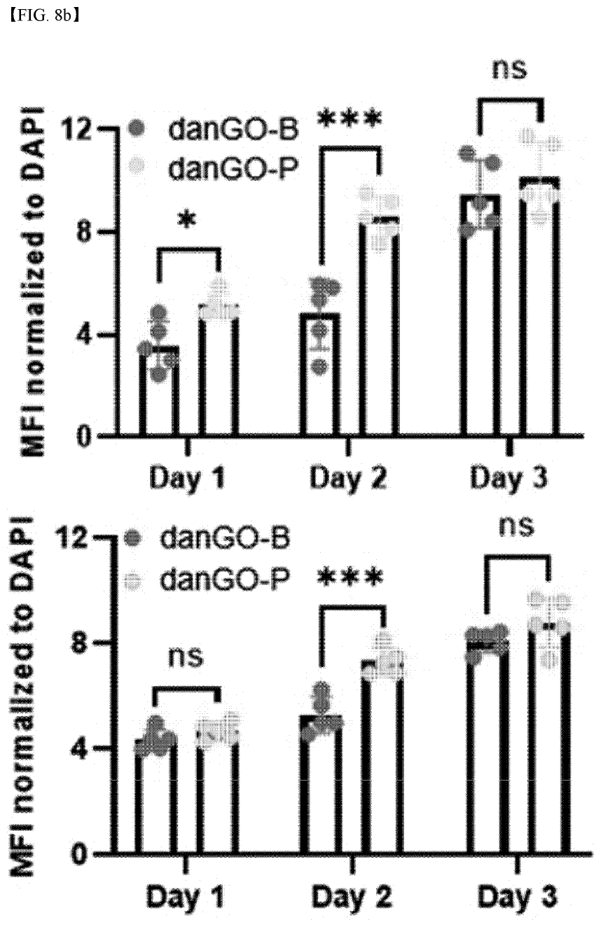

As a result, it was observed that the uptake of each graphene in colon cancer cells gradually increased until day 3 of treatment, and the uptake efficiency (rate) was shown to be higher in the experimental group than in the control group (FIG. 8). Therefore, regardless of the presence or absence of the bonded functional group, it had the characteristics of influx into cancer cell lines, and it was confirmed that the influx amount and rate of danGO-PEG were excellent.

### Example 3-3. Confirmation of the Growth Inhibitory Effect of Cancer Cells by Modified Nanographene Oxide

In order to confirm the growth inhibitory effect of cancer cells when the modified nanographene oxide is treated for a long period of time, the colony forming assay was performed after treatment with danGO-PEG and danGO-B. Specifically, 1,000 SW480 and SW620 cells were seeded, and after treating the experimental groups and control group with 10 μg/mL for about 2 weeks, they were stained with crystal violet.

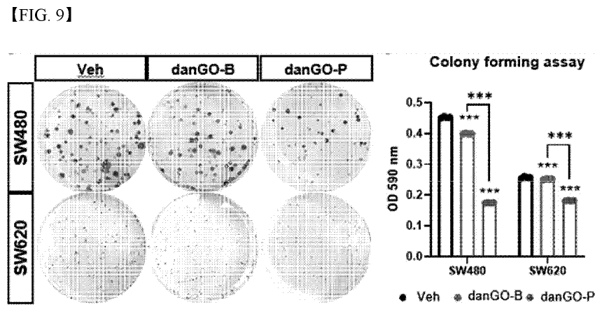

As a result, all of the experimental groups and the control group were found to have the growth inhibitory effect of cancer cells on both types of cancer cells, but the experimental groups had significantly more excellent growth inhibitory effects of cancer cells than the control group (FIG. 9). In particular, when the experimental groups were treated with SW480 cells for 2 weeks, the growth of cancer cells was significantly inhibited, and compared to no treatment and the treatment of the control group, this effect was confirmed to be approximately 2 folds (½ the number of cells). According to such results, the experimental groups according to the present invention were confirmed to have an excellent anticancer effect and cancer cell killing effect, as well as the growth inhibition effect of cancer cells in that despite the anticancer effect and cancer cell killing effect in the control group, the growth inhibition effect of cancer cells was investigated to be almost insignificant.

### Example 4. Effect of Modified Nanographene Oxide on Mitochondria in Cancer Cells

### Example 4-1. Confirmation of an Increase in the Number of Mitochondria in Cancer Cells by Modified Nanographene Oxide

When the analysis is performed through Mitotracker, it is possible to determine the total amount of mitochondrial in cells by tracking mitochondria, and thus, Mitotracker analysis was performed to confirm the effect of modified nano graphene oxide on increasing the number of mitochondria in cancer cells. Specifically, cancer cells were treated with 10 μg/mL of each graphene (danGO-PEG and danGO-B) for 48 hours, and 200 nM of Mitotracker was added to a culture medium (RPMI-1640), and after treating the cells for about 30 minutes in a CO₂ incubator at 37° C., quantitative analysis was performed by using Mitotracker (M22425, Invitrogen).

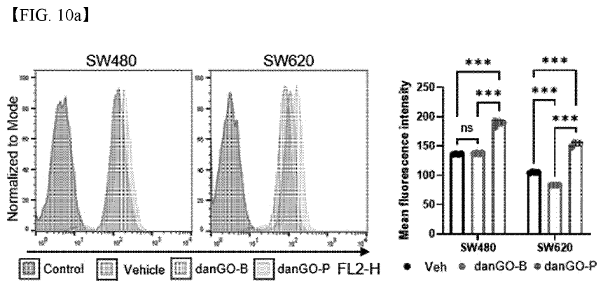

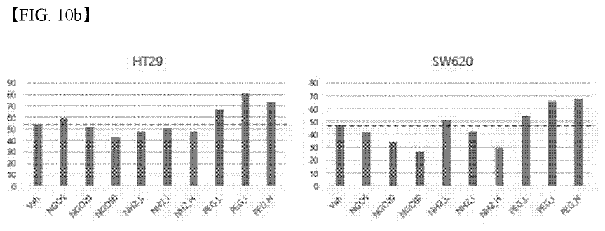

As a result, whereas the number of mitochondria in the control group was similar to that of the untreated group (SW480 cells) or decreased (SW620), the numbers of mitochondria in the two cancer cell lines significantly increased when the experimental groups were treated, and since it was found to be about 125% and about 150% in the SW620 cell line, it was confirmed that the effect of increasing the number of mitochondria in cancer cells by danGO-PEG was remarkably excellent (FIG. 10a).

In addition, quantitative analysis was performed by measuring the amount of mitochondria by treating NGO, NGO-NH₂ and NGO-PEG at various concentrations in the same experimental method.

As a result, as shown in FIG. 10b, it was confirmed that the amount of mitochondria significantly increased in the NGO-PEG group compared to the control group in HT29 (primary colon cancer cells) and SW620 (metastatic colon cancer cells). Through the above results, the amount of mitochondria is increased by the inhibition of mitophagy, and accordingly, conversion to metabolism that is capable of producing energy through oxidative phosphorylation by mitochondria in cancer cells is achieved, and thus, it was confirmed that it is possible to induce the apoptosis of cancer cells in hypoxia.

### Example 4-2. Confirmation of Mitochondrial Damage Effect in Cancer Cells by danGO-PEG

Since Mitotracker performed in Example 4-1 does not discriminate between healthy and damaged mitochondria and measures the total mass, the effect of danGO-PEG on mitochondrial damage in cancer cells cannot be confirmed. Therefore, in order to analyze the degree of mitochondrial damage in cancer cells, mitochondrial membrane potential change analysis and the ratio analysis of mitochondrial DNA and nuclear DNA were performed.

First of all, after SW480 and SW620 cancer cell lines were treated with 10 μg/mL of each graphene for 48 hours under the same conditions as in Example 4-1, changes in the mitochondrial membrane potential (mtMP) were measured by using JC-1 dye (T3168, Invitrogen). Upon JC-1 staining, healthy mitochondria appear as JC-1 aggregate (red), whereas damaged mitochondria appear as JC-1 monomer (green), which can confirm the degree of mitochondrial damage.

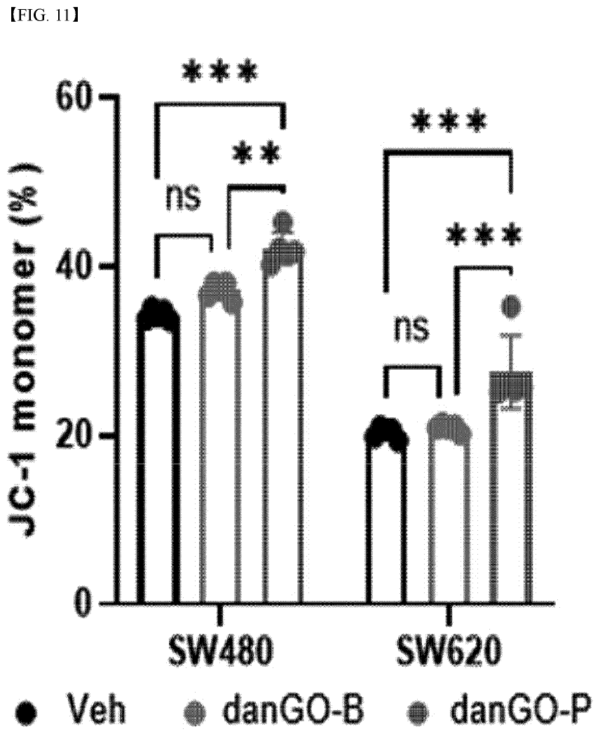

As a result of analyzing the ratio of JC-1 monomer in cancer cells that were treated with each graphene through flow cytometry, the ratio of JC-1 monomer in the PEG group was found to increase significantly, thereby inducing depolarization in mitochondria, and it was confirmed that damage occurred (FIG. 11).

In addition, as an indicator of mitochondrial damage, the ratio of mitochondrial DNA and nuclear DNA in cells was determined. Specifically, after DNA was extracted from each graphene-treated cancer cell, the expressions of mitochondrial-specific DNA (ND1; Forward: GGCTATATACAACTACGCAAAGGC, Reverse: GGTAGATGTGGCGGGTTTTAGG) and nuclear-specific DNA (SERPINA1; Forward: AAAGGTGGGACATTGCTGCT, Reverse: GATGCCCCACGAGACAGAAG) were compared.

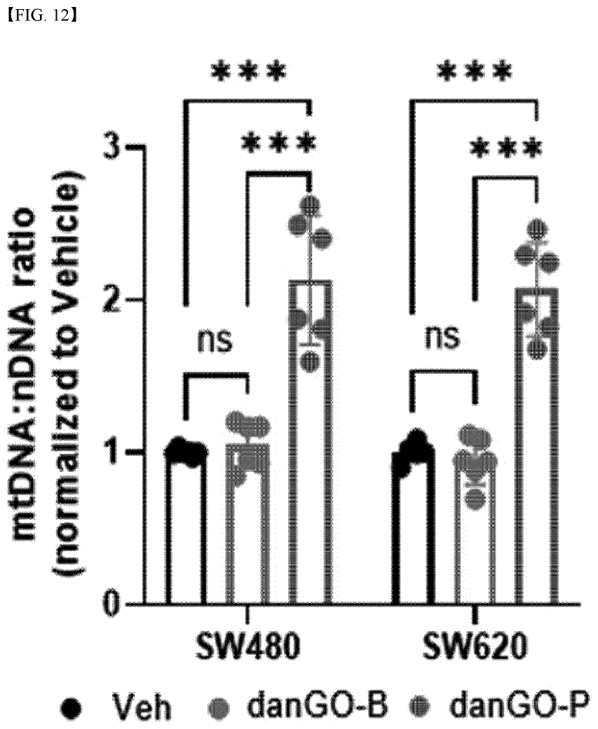

As a result, it was confirmed that mitochondria damaged by danGO-PEG increased in both colon cancer cell lines of SW480 and SW620 (FIG. 12). Specifically, the mitochondrial damage effect by the control group did not appear to occur, whereas the treatment with danGO-PEG showed that mitochondria were damaged by about 2 times or more in both types of cancer cells, which confirmed that it had an excellent anticancer effect due to mitochondrial damage.

### Example 4-3. Identification of ROS by Mitochondria in Cancer Cells Upon Treatment with danGO-PEG

When damaged mitochondria accumulate in cells, reactive oxygen species (ROS) are generated. Therefore, flow cytometry was performed to determine the degree of ROS caused by damaged mitochondria in cancer cells by danGO-PEG. Specifically, SW480 and SW620 were treated with 10 μg/mL of each graphene for 48 hours under the same conditions as in Example 4-1, and mitochondrial ROS in cancer cells was quantified through mitoSOX (M36008, Invitrogen) staining, and it was analyzed whether the total cellular ROS level increased through DCFDA assay (D399, Invitrogen).

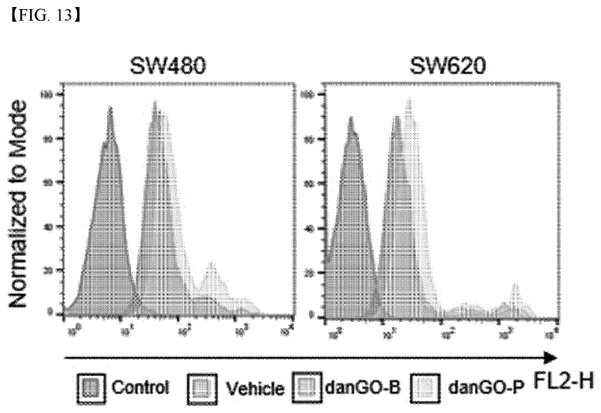

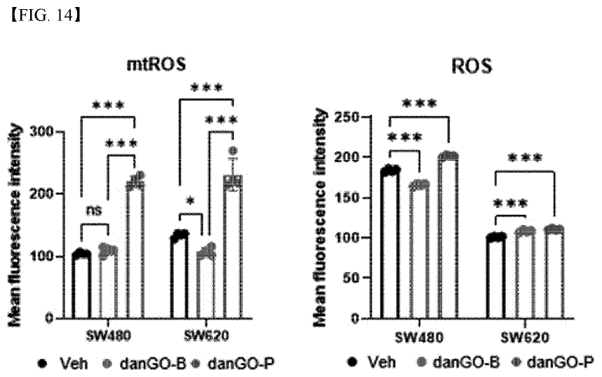

As a result, it was confirmed that not only ROS generation of damaged mitochondria increased significantly in the PEG group (FIG. 13), but also the total cellular ROS levels increased (FIG. 14). Specifically, the level of mitochondrial and total ROS generation by the control group was similar to or rather reduced than that of the untreated group, whereas danGO-PEG was found to be about twice as high as those of the untreated group and control group in ROS generation of damaged mitochondria, and it was also confirmed that the increase effects were excellent in the total cell ROS levels compared to the untreated group and the control group.

### Example 4-4. Confirmation of Mitochondrial Respiratory Metabolism Changes in Cancer Cells by danGO-PEG

In order to determine the effect of danGO-PEG on mitochondrial respiration and metabolism (oxidative phosphorylation) in cancer cells, SW480 and SW620 were treated with 10 μg/mL of each graphene for 48 hours under the same conditions as in Example 4-1, and then, changes in the respiratory metabolism of mitochondria were confirmed. Specifically, after mitochondria in cancer cells were isolated by using a mitochondria isolation kit (89874, Thermofisher Scientific), Western blot was performed. The expression of a respiratory chain complex involved in oxidative phosphorylation in the mitochondrial fraction was determined by using OXPHOS antibody cocktail (ab110411, Abcam). In this case, the mitochondrial marker TOMM20 (ab56783, Abcam) was used as a loading control.

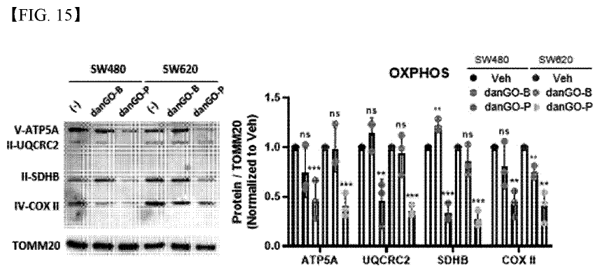

As a result, it was confirmed that the expression of each complex in the PEG group was significantly decreased compared to the untreated group and the control group (FIG. 15). Specifically, as a result of Western blot, when danGO-PEG was treated, a decrease in complex expression in each cancer cell line was not only visually confirmed (left graph in FIG. 15), but also it was confirmed to be significantly superior on the graph compared to the control group (right graph of FIG. 15).

### Example 4-5. Confirmation of Cytochrome c Release Effect of Mitochondria in Cancer Cells by danGO-PEG

It is known that various pathways related to apoptosis are activated as cytochrome C which is present in mitochondria is released into the cytoplasm when cells die (apoptosis). Therefore, the expression of cytochrome C in mitochondria and cytosol in cells after danGO-PEG treatment was analyzed. Specifically, SW480 and SW620 were treated with each graphene at 10 μg/mL for 48 hours under the same conditions as in Example 4-1, and then, mitochondria and cytoplasm were isolated, respectively, and the expression of cytochrome C (sc13560, Santa Cruz) was determined through Western blot. In this case, HSP60 (12165, Cell Signaling Technology) was used as a loading control for the mitochondrial fraction, and a-tubulin (3873, Cell Signaling Technology) was used as a loading control for the cytosol fraction.

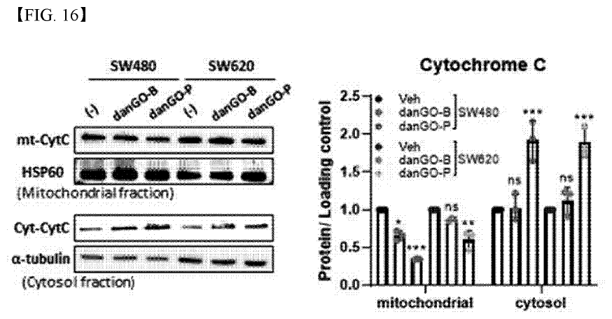

As a result, it was confirmed that when danGO-PEG was treated, the expression of cytochrome C in the mitochondria was reduced by about 0.3 to 0.5 times compared to the control group, whereas the expression of cytochrome C in the cytoplasm was significantly increased by about 2 times (FIG. 16). This suggests that the apoptosis of cancer cells by mitochondrial damage progresses, and cytochrome C in mitochondria is released into the cytoplasm.

### Example 5. Mitophagy Regulation in Cancer Cells by danGO-PEG

Mitophagy is classified into the NIX-dependent pathway and the PINK1/Parkin-dependent pathway. In the NIX-dependent pathway, NIX expressed in mitochondria directly binds with the phagophore LC3, which is a double membrane that surrounds and separates cytoplasmic components during macroautophagy, thereby forming autophagosomes, and decomposition takes place through binding with lysosomes. On the other hand, in the PINK1-dependent pathway, PINK1 expressed in mitochondria recruits Parkin (Ubiquitin ligase) and ubiquitin to induce the ubiquitination of damaged mitochondria, and when this complex binds to the phagophore, autophagosome form, and as a result, decomposition occurs.

Therefore, the expressions of NIX, BNIP3 and PINK1 were analyzed to determine the mitophagy-regulating effect of modified nanographene oxide including danGO-PEG and related mechanisms in cancer cells.

### Example 5-1. Confirmation of Mitophagy Regulation of danGO-PEG and danGO-B

After SW480 and SW620 were treated with each graphene 10 μg/mL for 48 hours under the same conditions as in Example 4-1, changes in the mRNA expression of NIX (NBP1-88558, Novus Biologicals), which is a receptor-mediated mitophagy in cancer cells, were determined through quantitative PCR (Forward: CTACCCATGAACAGCAGCAA, Reverse: ATCTGCCCATCTTCTTGTGG).

In addition, the expression levels of NIX, BNIP3 (sc 56167, Santa Cruz), which is another receptor-mediated mitophagy, and PINK1 (BC100-494, Novus Biologicals), which is a PINK1/Parkin-dependent mitophagy, were analyzed through Western blot (Loading control: GAPDH, 97166, Cell Signaling Technology), and finally, fluorescence immunostaining analysis was performed.

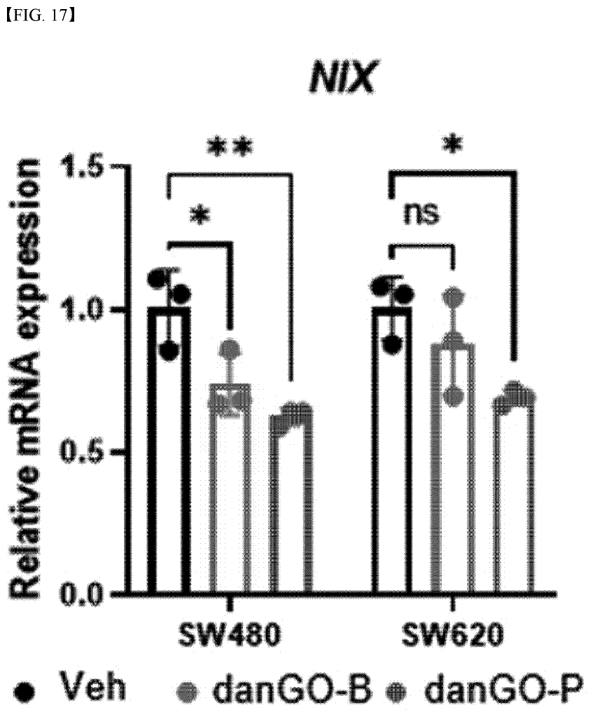

As a result, it was found that the mRNA expression of NIX was reduced statistically significantly in the PEG group (FIG. 17). In particular, it was confirmed that the expression of NIX was about 0.6 times higher than that of the untreated group in the SW480 cell line and about 0.7 times higher than that of the untreated group in the SW620 cell line.

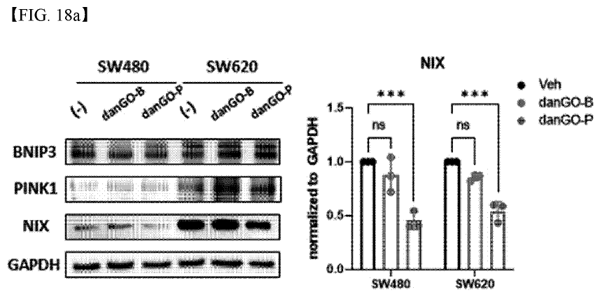

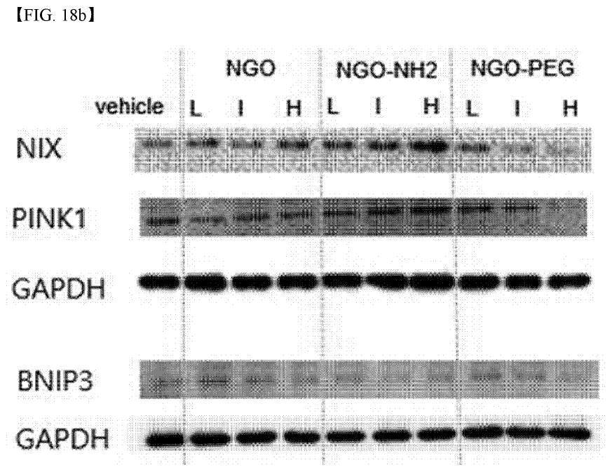

As a result of Western blot analysis, it was confirmed that the expression of NIX was significantly reduced when the PEG group was treated compared to the untreated group and the control group. On the other hand, the expression of BNIP3 tended to increase rather in the PEG group and the control group than in the untreated group, which was shown to be similar to the expression of PINK1 (FIG. 18).

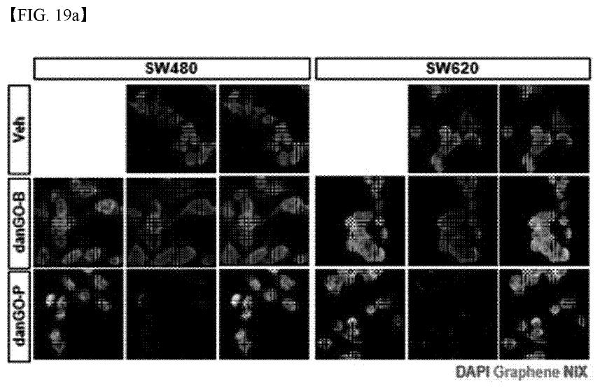

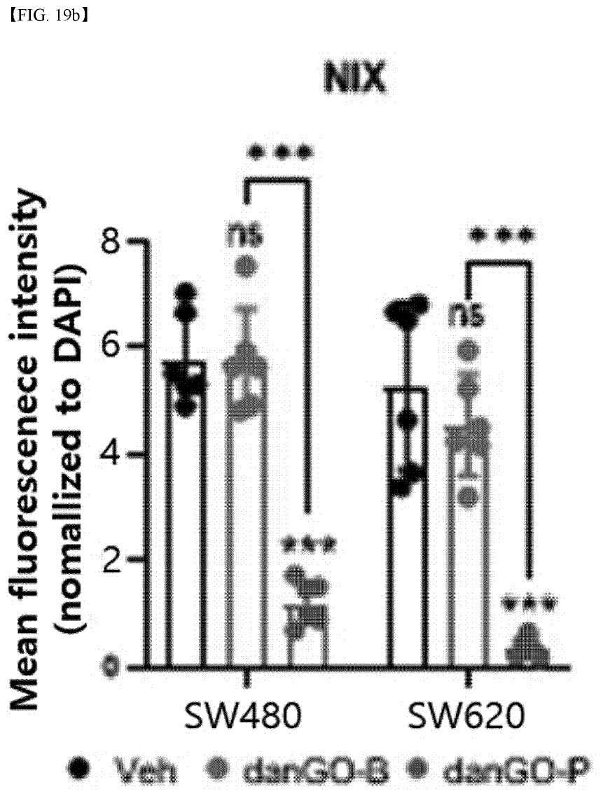

Finally, as a result of fluorescence immunostaining, the expression of NIX in cancer cells was significantly reduced in the PEG group, and particularly, the co-localization of graphene particles and NIX that were introduced into cells was greatly reduced (FIGS. 19a and 19b).

### Example 5-2. Confirmation of Mitophagy Regulation According to Various Concentrations of NGO, NGO-NH₂ and NGO-PEG

In the same manner as in Example 5-1, various concentrations of NGO, NGO-NH₂ and NGO-PEG were treated to perform Western blot for PINK1 (PTEN Induced Kinase 1, #BC100-494, Novus Biologicals), NIX (Nip3-like protein X, #12396, Cell signaling technology) and BNIP3 ((Bcl2/adenovirus E1B 19 kDa protein-interacting protein 3, #sc-56167, Santa Cruz Biotechnology). In this case, the concentrations of NGO, NGO-NH₂ and NGO-PEG were treated at 5, 20 and 50 μg/mL, respectively.

As a result, as shown in FIG. 18b, it was confirmed that only the NGO-PEG group significantly decreased the amount of the marker compared to the control group.

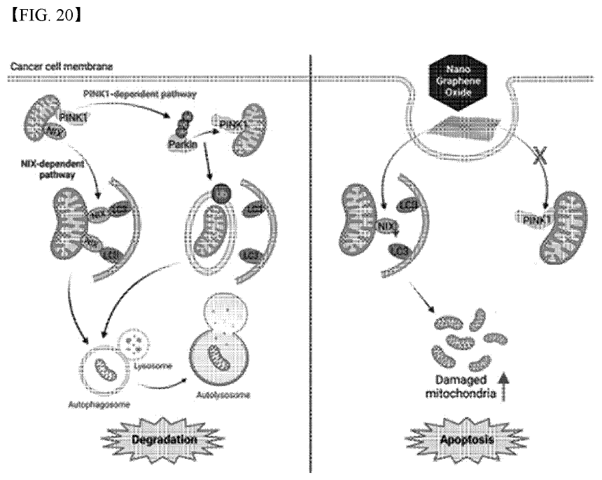

According to these results, since the NIX-dependent pathway was reduced when danGO-PEG was treated, the graphene particles entered the cells through endocytosis in the cell membrane, and then, they targeted mitochondria to reduce NIX, which reduced binding to LC3 such that the decomposition of damaged mitochondria did not occur, and it was investigated that this affected the energy metabolism of cancer cells, thereby leading to cell apoptosis (FIG. 20). That is, danGO-PEG exhibits an anticancer effect by specifically inhibiting the activity of NIX to induce autophagy within mitochondria.

### Example 6. Control of Autophagy in Cancer Cells of Modified Nanographene Oxide

### Example 6-1. Autophagic Flux in Cancer Cells of danGO-PEG and danGO-B

Autophagy flux, which is one of the important mechanisms that regulate the apoptosis of cancer cells together with mitophagy, was analyzed. Specifically, Western blotting was performed to determine the protein expressions of LC3 and p62 that are involved in autophagy flow.

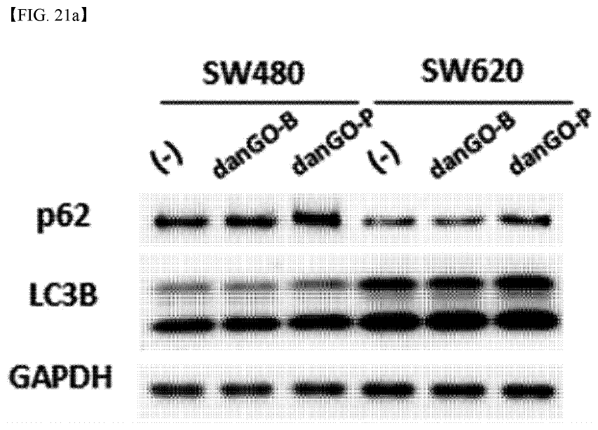

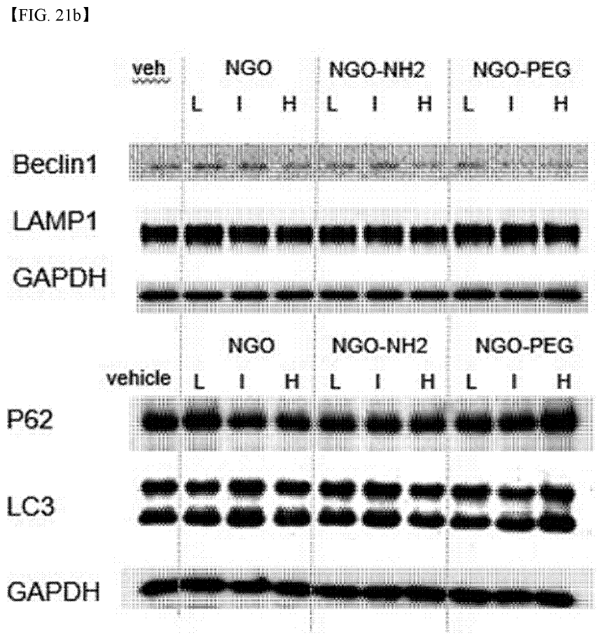

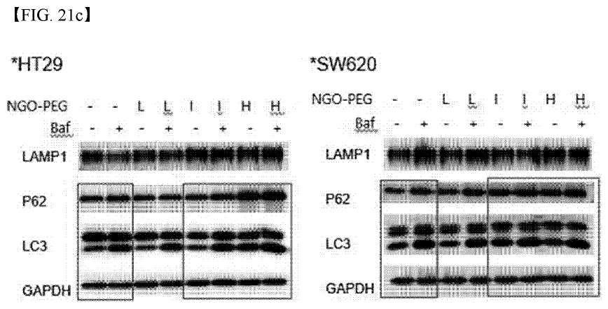

As a result, as the expressions of LC3B II (autophagy initiation, autophagosome formation) and p62 increased simultaneously in the PEG group, autophagosome formation occurred normally, but fusion with lysosome was not performed well, and thus, it was analyzed that p62 was not degraded (FIG. 21). Therefore, it was suggested that even though mitophagy is activated through the binding of NIX and LC3, there is a possibility that the degradation of finally damaged mitochondria may not occur by suppressing the fusion between mitophagosome and lysosome. In other words, as the NIX-dependent mitophagy pathway is inhibited by the PEGylated graphene of the present invention, damaged mitochondria in cancer cells are not removed and accumulated in cells, thereby disrupting mitochondrial equilibrium and inhibiting metabolism for energy production in cancer cells, and thus, the apoptosis of cancer cells may be induced.

### Example 6-2. Autophagy Flow in Cancer Cells According to Various Concentrations of NGO, NGO-NH₂ and NGO-PEG

After cancer cells were treated with NGO, NGO-NH₂ and NGO-PEG at various concentrations (NGO 5, 20 and 50 μg/mL, NGO-NH₂ 4, 20 and 40 μg/mL, NGO-PEG 5, 20 and 40 μg/mL), the samples were lysed with Pro-Prep (Intron Biotechnology Co., Sungnam, Republic of Korea) to extract proteins from the tissue. The obtained protein samples were separated by 10% SDS-PAGE (sodium dodecyl sulfate polyacrylamide gel electrophoresis) and transferred to a nitrocellulose membrane. After blocking with 3% BSA (Bovine Serum Albumin) solution, the proteins on the membrane were incubated at 4° C. for at least 12 hours with primary antibodies of Beclin (#3495S, Cell Signaling Technology), LAMP1 (#sc20011, Santa Cruz Biotechnology), p62 (#610832, BD Bioscience) and LC3B II (#NB100-2220, Novus Biologicals), and incubated with a secondary antibody (goat anti-mouse IgG HRP-conjugated, #G21040, Invitrogen; goat anti-rabbit IgG HRP-conjugated, #G21234), Invitrogen). Protein and antibody complexes were detected by using ECL Western blotting Detection Reagent and Analysis System. The results that were confirmed by Western blotting are shown in FIG. 21b.

In addition, the drug bafilomycin inhibits the fusion of autophagosome and lysosome, and experiments were conducted with and without the drug to determine the expression of proteins involved in autophagy flow. In more detail, HT269 and SW620 were treated with NGO-PEG at three concentrations (NGO-PEG 5, 20 and 40 μg/mL), and by varying whether bafilomycin was treated or not, the same procedure as the Western blot was performed. The results that were confirmed by Western blot are shown in FIG. 21c.

As shown in FIG. 21b, it was confirmed that the expressions of LC3B II (autophagy initiation, autophagosome formation), which serves to recruit autophagosome members, and p62, which is an indicator protein of autophagy, were simultaneously increased in the NGO-NH₂ group and the NGO-PEG group. In addition, it can be confirmed that the expression of Beclin1, which is a protein involved in autophagy, was decreased, and the expression of LAMP1 (Lysosomal Associated Membrane Protein 1), which is involved in autophagy as a lysosome-associated membrane protein, was increased.

In addition, as shown in FIG. 21c, it was confirmed that the expressions of LC3B II, p62 and LAMP1 were increased in the NGO-PEG group compared to the vehicle group, regardless of whether bafilomycin was treated or not. Since an increase in LC3B II indicates the formation of autophagosomes and an increase in LAMP1 indicates the formation of lysosomes, this indicates a possibility of binding to lysosomes and autophagosomes. However, considering that p62 is degraded by autophagy, it can be seen that an increase in p62 does not facilitate fusion between autophagosome and lysosome.

Accordingly, it can be confirmed that NGO-NH₂ or NGO-PEG inhibits fusion between autophagosomes and lysosomes. That is, in the NGO-NH₂ group and the NGO-PEG group, it was suggested that cancer cells could be killed by controlling the autophagy flow mechanism.

### Example 7. Verification of In Vivo Cancer Occurrence and Progression Mitigation Effect Upon Oral Administration of Nanographene Oxide and Modified Structure Thereof

### Example 7-1. Confirmation of In Vivo Cancer Occurrence and Progression Mitigation Effect of Nanographene Oxide Structure

It was determined whether orally administering the nanographene structure or a modified structure thereof has an effect on cancer occurrence and the mitigation of progression in vivo. Specifically, according to Table 1, rats that were not induced with colon cancer (sham group) and rats that were induced with colon cancer and administered with nanographene oxide (danGO group) were used, and rats that were induced with colon cancer were used as a negative control group (vehicle group), and as a positive control group, rats that were administered with 5-fluorouracil (5-FU), which is an anticancer drug, were prepared. In the negative control group, colon cancer was induced by using Azoxymethane and Dextran sodium sulfate (DSS). In the danGO group which was the experimental group, danGO was orally administered at a dose of 250 μg/mouse 5 times every 3 days to rats that were induced with colon cancer.

TABLE 1

| | Untreated | Negative | Experimental | Positive |
|---|---|---|---|---|
| | Group | Control Group | Group | Control Group |
| Classification | sham group | vehicle group | danGO group | 5-fu |
| Content | Control group not | Saline | danGO | 5-FU |
| | induced with | administration | administration | administration |
| | colon cancer | group | group | group |

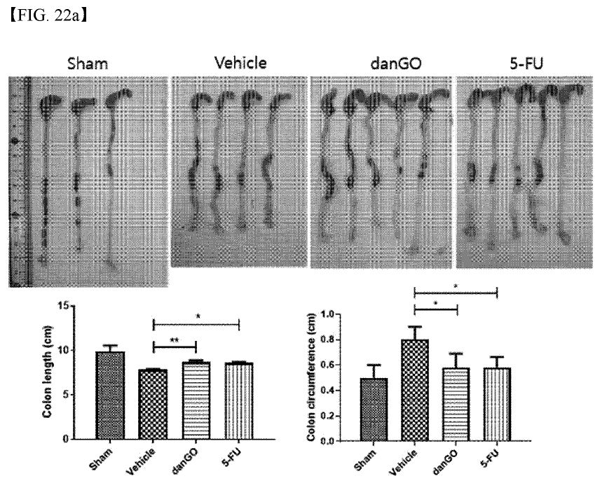

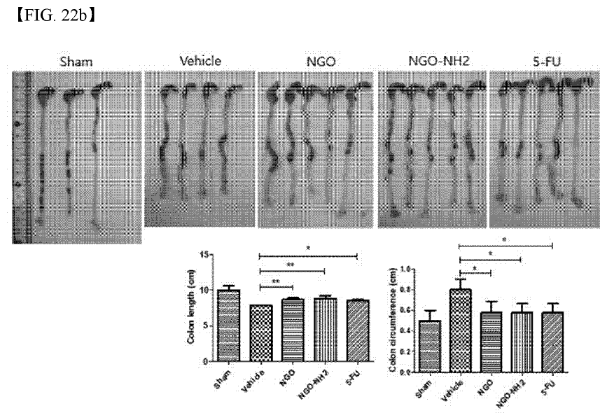

First of all, the effect of the nanographene structure on the length of the colon was confirmed. The length of the colon is affected by inflammation and shows a tendency to decrease as inflammation occurs, and whereas the length of the colon was significantly decreased and the diameter increased in the vehicle group compared to the sham group, the length of the colon was preserved statistically significantly in the danGo group and 5-FU group compared to the vehicle group, and thus, the effect of alleviating the disease was confirmed (FIG. 22a).

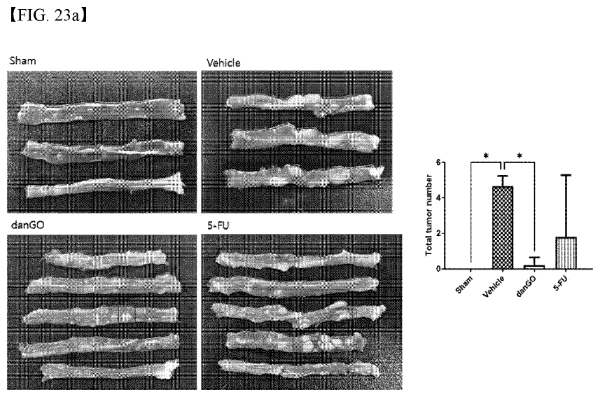

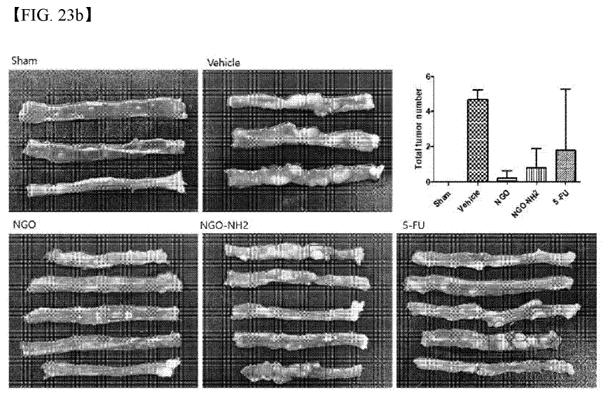

Next, the effect on solid tumors in the lumen of the colon was confirmed. As a result of determining the lumen of the colon, an average of 5 solid cancers were observed in the mid-distal region of the colon in the vehicle group, but an average of 1 to 2 solid cancers was observed in the danGO group (FIG. 23a). It was analyzed that nanographene oxide exhibits an excellent anticancer effect in that it has a stronger anticancer effect than 5-FU, which is a clinically used anticancer drug for colon cancer patients.

### Example 7-2. Confirmation of In Vivo Cancer Occurrence and Progression Mitigation Effect of Modified Nanographene Oxide Structure

In the same manner as in Example 7-1, the effect of modified nanographene oxide, NGO-NH₂, on in vivo cancer occurrence and progression mitigation was confirmed.

TABLE 2

| | | Negative | Positive | | |
|---|---|---|---|---|---|
| | Untreated | Control | Control | Experimental Group | |
| | Group | Group | Group | | NGO—NH₂ |
| Classification | sham group | vehicle group | 5-FU group | danGO group | group |
| Content | Control group | Saline | 5-FU | danGO | NGO—NH₂ |
| | not induced | administration | administration | administration | administration |
| | with colon | group | group | group | group |
| | cancer | | | | |

As a result of administering the modified nanographene oxide, as shown in FIG. 22b, whereas the length of the colon was significantly decreased and the diameter was increased in the vehicle group compared to the sham group, it was confirmed that the disease was significantly alleviated in the NGO group, NGO-NH₂ group and 5-FU group compared to the vehicle group.

Next, the effect on solid tumors in the lumen of the colon was determined.

As a result, as shown in FIG. 23b, when examining the lumen of the colon, it was confirmed that an average of 5 solid cancers were observed in the mid-distal region of the colon in the vehicle group, but an average of 1 to 2 solid tumors was observed in the NGO group and the NGO-NH₂ group. This is less than the number of solid tumors in the 5-FU group. Therefore, it can be confirmed that NGO and NGO-NH₂ are more effective than the anticancer agent (5-FU) in suppressing the occurrence of cancer.

Next, the spleen was separated and the size and weight were measured in order to determine the effect on the degree of systemic inflammation. The spleen is an important lymphoid organ, accounting for about 25% of the body weight of the lymphatic organ. The spleen is in charge of the immune function of removing bacteria or foreign proteins that invade the body, and removes various blood cells including aged red blood cells and platelets and cells bound to immunoglobulins. The spleen can enlarge in response to infection or inflammation.

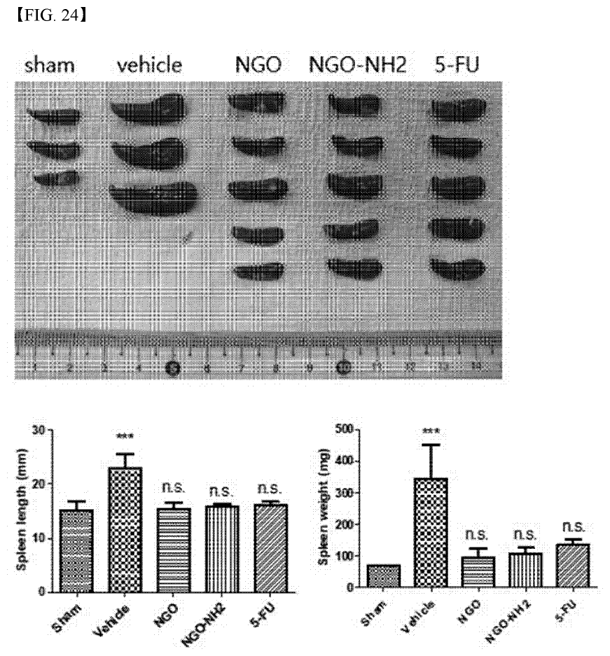

As shown in FIG. 24, whereas the length and weight of the spleen were increased significantly in the vehicle group compared to the sham group, it was confirmed that the inflammatory response was alleviated to a level similar to that of the sham group in the groups administered with NGO, NGO-NH₂ and a positive control group anticancer drug.

Through the above results, it was confirmed that the nanographene oxide structures (NGO, NGO-NH₂) showed a stronger anticancer effect than 5-FU, which is a clinically used anticancer drug in colon cancer patients, and it was confirmed that the occurrence of cancer was significantly reduced in the NGO and NGO-NH₂ administration groups compared to the control group. Through this, it can be confirmed that the nanographene oxide or nanographene oxide with modified functional groups can function as a potential anticancer agent.

The description of the present invention described above is for illustrative purposes, and those skilled in the art will understand that it can be easily modified into other specific forms without changing the technical spirit or essential features of the present invention. Therefore, the exemplary embodiments described above should be understood as illustrative in all respects and not restrictive.

### INDUSTRIAL APPLICABILITY

The composition for preventing and treating cancer through mitochondrial metabolism regulation, including a nanographene structure, has been confirmed that a specific inhibitory activity on the NIX-dependent pathway among the mitophagy mechanisms in cancer cells inhibits mitochondrial degradation, and thus, damaged mitochondria accumulate in the cancer cells, and accordingly, apoptosis of the cancer cells is induced as a result. Therefore, since the composition according to the present invention exhibits excellent anticancer effects that can be applied to various diseases related to the accumulation of abnormal cells, without causing resistance or toxicity, it is expected that it can be advantageously used as a cancer prevention or treatment agent or an anticancer treatment adjuvant, and thus, the industrial applicability of the present invention is acknowledged.

1-46. (canceled)

47. A nanographene oxide variant exhibiting any one or more of the following characteristics:
1) it has a circular plate shape;
2) as a result of Raman spectroscopy analysis, it has a carbon-derived material-specific Raman shift; and
3) as a result of Raman spectroscopy analysis, graphene active groups —OH, —C—H and C═O are confirmed.

48. The nanographene oxide variant of claim 47, wherein the nanographene oxide variant is a PEGylated nanographene oxide or an aminated nanographene oxide in which PEG (polyethylene glycol) or NH₂ (amino group) is bonded to a nanographene oxide.

49. The nanographene oxide variant of claim 48, wherein the PEGylated nanographene oxide variant exhibits any one or more of the following characteristics:
1) the zeta potential of graphene ranges from −25.0 to −21.0 mV; and
2) it has a particle size of approximately 10 nm.

50. The nanographene oxide variant of claim 48, wherein the aminated nanographene oxide variant exhibits the following characteristic:
1) it has a diameter of 24.4 to 38.8 nm.

51. A method for preventing, ameliorating or treating cancer comprising administering the nanographene oxide variant of claim 47 to a subject in need thereof.

52. The method of claim 51, wherein the nanographene oxide variant inhibits mitochondrial mitophagy.

53. The method of claim 51, wherein the nanographene oxide variant specifically inhibits NIX.

54. The method of claim 51, wherein the nanographene oxide variant does not inhibit BNIP3 and PINK1.

55. The method of claim 51, wherein the nanographene oxide variant is administered at a concentration of 0.01 to 200 μg/mL.

56. The method of claim 51, wherein the cancer is one or more selected from the group consisting of thyroid cancer, cervical cancer, brain cancer, lung cancer, ovarian cancer, bladder cancer, kidney cancer, liver cancer, pancreatic cancer, prostate cancer, skin cancer, tongue cancer, breast cancer, uterine cancer, stomach cancer, rectal cancer, colon cancer, blood cancer, bone cancer, oral cancer, pharynx cancer, laryngeal cancer and colon carcinoma.

57. A method for supplementing anticancer therapy comprising administering the nanographene oxide variant of claim 47 to a subject in need thereof.

58. The method of claim 57 wherein the cancer is one or more selected from the group consisting of thyroid cancer, cervical cancer, brain cancer, lung cancer, ovarian cancer, bladder cancer, kidney cancer, liver cancer, pancreatic cancer, prostate cancer, skin cancer, tongue cancer, breast cancer, uterine cancer, stomach cancer, rectal cancer, colon cancer, blood cancer, bone cancer, oral cancer, pharynx cancer, laryngeal cancer and colon carcinoma.

59. A method for inhibiting mitophagy in a cancer cell comprising administering the nanographene oxide variant of claim 47 to the cancer cell.


SEQUENCE LISTING (6 sequences)

SEQ ID NO: 1 - DNA, 24 bp, synthetic construct
  source 1..24: mol_type = other DNA
ggctatatacaactacgcaaaggc

SEQ ID NO: 2 - DNA, 22 bp, synthetic construct
  source 1..22: mol_type = other DNA
ggtagatgtggcgggttttagg

SEQ ID NO: 3 - DNA, 20 bp, synthetic construct
  source 1..20: mol_type = other DNA
aaaggtgggacattgctgct

SEQ ID NO: 4 - DNA, 20 bp, synthetic construct
  source 1..20: mol_type = other DNA
gatgccccacgagacagaag

SEQ ID NO: 5 - DNA, 20 bp, synthetic construct
  source 1..20: mol_type = other DNA
ctacccatgaacagcagcaa

SEQ ID NO: 6 - DNA, 20 bp, synthetic construct
  source 1..20: mol_type = other DNA
atctgcccatcttcttgtgg